(19) United States
(12) Patent Application Publication
(10) Pub. No.: US 2010/0144868 A1
(43) Pub. Date: Jun. 10, 2010

(54) Novel Neuroprotective Compounds and Uses Thereof

(75) Inventors: Michael Gozin, Tel-Aviv (IL); Howard L. Weiner, Brookline, MA (US); Alon Monsonego, Moshav Nir-Banim (IL); Amnon Bar-Shir, Ramat-Hakovesh (IL); Yoni Engel, RaAnana (IL); Dan Frenkel, Rechovot (IL)

Correspondence Address: MARTIN D. MOYNIHAN d/b/a PRTSI, INC., P.O. BOX 16446, ARLINGTON, VA, 22215, US

(73) Assignees: Ramot At Aviv University Ltd., Tel-Aviv (IL); The Brigham and Women's Hospital Inc., Boston, MA (US)

(21) Appl. No.: 11/795,808

(22) PCT Filed: Jan. 22, 2006
(86) PCT No.: PCT/IL06/00092

§ 371 Date: Dec. 3, 2009

Related U.S. Application Data
(60) Provisional application No. 60/645,001, filed on Jan. 21, 2005.

(51) Int. Cl. A61K 31/27; C07C 271/24; A61P 9/00; A61P 35/00; A61P 3/00; A61P 11/06; A61P 25/00

(52) U.S. Cl. 514/480; 560/115; 977/735

(57) ABSTRACT

Disclosed are novel hybrid compounds having a fullerene core residue, one or more bioavailability enhancing moieties and one or more glutamate receptor ligand residues, whereby the bioavailability enhancing moiety allow the compound to reach an effective concentration in physiological media and pass the blood-brain barrier, as defined in the specification. Also disclosed are pharmaceutical compositions containing these hybrid compounds and uses thereof as antioxidants and/or neuroprotective agents for the treatment of medical conditions associated with oxidative stress and/or neural damage, such as, for example, neurological diseases, disorders and trauma, and hence in the treatment of CNS-associated diseases, disorders and trauma, as well as to uses thereof as antiviral, antibacterial, antiglycemic, antiarrhythmic, antidepressant and antitumor agents.

### FIELD AND BACKGROUND OF THE INVENTION

The present invention relates to novel hybrid compounds and uses thereof and, more particularly, to fullerene-adamantane hybrid compounds and uses thereof as antioxidants and/or neuroprotective agents for the treatment of medical conditions associated with oxidative stress and/or neural damage, such as, for example, neurological diseases, disorders and trauma, and hence in the treatment of CNS-associated diseases, disorders and trauma, as well as to uses thereof as antiviral, antibacterial, antiglycemic, antiarrhythmic, antidepressant and antitumor agents.

Oxidative stress may be considered as a disturbance in the equilibrium status of pro-oxidant/anti-oxidant systems in intact cells, and may result from a number of different oxidative challenges, including radiation, metabolism of environmental pollutants and administered drugs, as well as immune system response to disease or infection. When oxidative stress occurs, the pro-oxidant systems outbalance those of the anti-oxidant, which may result in oxidative damage to cell components including lipids, proteins, carbohydrates, and nucleic acids. Mild, chronic oxidative stress may alter the anti-oxidant systems by inducing or repressing proteins that participate in these systems, and by depleting cellular stores of anti-oxidant materials such as glutathione and Vitamin E. Severe oxidative stress may ultimately lead to cell death.

Oxidative stress therefore involves reactive oxygen species (ROS), which have been implicated in the development of many heart and central nervous system (CNS) dysfunctions. Ischemia/reperfusion insults to these organs are among the leading causes of mortality in humans. These insults are caused by complete or partial local occlusions of heart and brain vasculature, by heart stroke or attack, and by cerebral attacks and trauma to the brain. In addition, ROS are involved in artherosclerotic lesions, in the evolution of various neurodegenerative diseases, and are also produced in association to epileptic episodes, in inflammation, in the mechanisms of action of various neurotoxicants, or as side-effects of drugs. Hence, antioxidative agents and drugs constitute a highly sought after target in contemporary drug development and pharmaceutical research.

Chronic degenerative changes, as well as delayed or secondary neuronal damage following direct injury to the CNS, may result from pathologic changes in the brain's endogenous neurochemical systems. Although the precise mechanisms mediating secondary damage are poorly understood, post-traumatic neurochemical changes may include overactivation of neurotransmitter release or re-uptake, changes in presynaptic or postsynaptic receptor binding, or the pathologic release or synthesis of endogenous factors. The identification and characterization of these factors and of the timing of the neurochemical cascade after CNS injury provides a window of opportunity for treatment with pharmacologic agents that scavenge ROS, modify synthesis, release, receptor binding, or physiologic activity of neurotransmitters and other endogenous factors with subsequent attenuation of neuronal damage and improvement in outcome. A number of studies have suggested that modification of post-injury events through pharmacologic intervention can promote functional recovery in both a variety of animal models and clinical CNS injury. Pharmacologic manipulation of endogenous systems by such diverse pharmacologic agents as anticholinergics, excitatory amino acid antagonists, including specifically N-methyl-D-aspartate (NMDA) receptor antagonists, endogenous opioid antagonists, catecholamines, serotonin antagonists, modulators of arachidonic acid, antioxidants and free radical scavengers, steroid and lipid peroxidation inhibitors, platelet activating factor antagonists, anion exchange inhibitors, magnesium, gangliosides, and calcium channel antagonists have all been suggested to potentially improve functional outcome after brain injury.

The pathogenesis of a diverse group of neurological disorders has been linked to excessive activation of excitatory amino acid receptors such as the NMDA receptor. These disorders include epilepsy, focal and global ischemia, CNS trauma, and various forms of neurodegeneration including multiple sclerosis (MS), Huntington's chorea, Parkinson's disease and Alzheimer's disease. There has been extensive effort invested in the development of excitatory amino acid receptor antagonists as therapeutic agents. Hence, NMDA antagonistic and therapeutic agents also constitute a highly sought after target in contemporary pharmaceutical research.

Fullerenes are members of a class of carbon molecule having an even number of carbon atoms arranged in the form of a cluster, such as a closed hollow cage, typically spheroid like a soccer ball, wherein the carbon-carbon bonds define a polyhedral structure. The carbon clusters contain an even amount of carbon atoms, generally ranging from 20-120 carbon atoms. The majority of the fullerenes produced are C₆₀ and C₇₀. The most abundant species to date is the C₆₀ molecule, known as buckminsterfullerene, or “buckyball”, named after R. Buckminster Fuller, the architect of the geodesic dome. C₆₀ consists of 12 pentagons and 20 hexagons and is classified as an icosahedron, the highest symmetry structure possible. Fullerenes are characterized as “radical sponges” because of their unique cage structure, which allows them to interact effectively with free radicals, hence fullerenes are known for their antioxidative activity.

During recent years research in the field of water-soluble C₆₀ fullerene derivatives has significantly increased due to a broad range of biological activity that was found for these compounds. This includes antioxidant and neuroprotective properties, inhibitory activity for various enzymes, antiviral and antibacterial properties, compounds with the potential to be developed as anticancer drugs and imaging diagnostic agents. One of the well-established approaches to overcome the lack of fullerenes solubility in aqueous solutions is by fullerenes' chemical modifications with polar groups such as polyols [Smith, P. F. and Darlington, C. L., Multiple Sclerosis 1999, 5(2), 110-120], carboxylates [Romrell, J. et, al., Exp. Opin. Pharmacotherapy 2003, 4(10), 1747-1761; and Tariot, P. N., J. Am. Med. Assoc. 2004, 291(14), 1695], polyethers [Koch, H. J. et al., Curr Pharm. Design 2004, 10(3), 253-259; and Danysz, W. et al., Neuroscience Biobehavioral Rev. 1997, 21(4), 455-468] and dendrons [Le, D. A. and Lipton, S. A., Drugs Aging, 2001, 18(10), 717-724; and Rison, R. A. and Stanton, P. K., Neuroscience Biobehavioral Rev. 1995, 19(4), 533-52].

Further development of this concept led to construction of hybrid systems in which a variety of functional moieties such as peptides [Ametamey, S. M. et al., J. Receptor Sig. Transduction Res. 1999, 19(1-4), 129-141; and Bressan, R. A. and Pilowsky, L. S., Eur. J. Nuc. Med. 2000, 27(11), 1723-31], oligonucleotides, porphyrins, DNA-binding and protein-binding fragments were attached to fullerene core through biocompatible linkers. Such dyad systems could amplify or alter biochemical characteristics of their components, or even produce compounds with new biological properties.

Water-soluble derivatives of buckminsterfullerene (C₆₀) derivatives constitute a unique class of compounds with potent therapeutic antioxidant properties. Studies on one class of these compounds, the malonic-acid-C₆₀ derivatives (carboxyfullerenes), indicated that they are capable of eliminating both superoxide anion and H₂O₂, and were effective inhibitors of lipid peroxidation, as well. Carboxyfullerenes demonstrated robust neuroprotection against excitotoxic, apoptotic and metabolic insults in cortical cell cultures, as disclosed, for example, in U.S. Pat. No. 6,265,443. They were also capable of rescuing mesencephalic dopaminergic neurons from both monopotassium phosphate (MPP⁺) and 6-hydroxydopamine-induced degeneration. Ongoing studies in other animal models of CNS disease states suggest that these novel antioxidants are potential neuroprotective agents for other neurodegenerative disorders, including Parkinson's disease [Dugan L. L. et al., Parkinsonism Relat. Disord., 2001, 7(3), pp. 243-246].

Further use of fullerenes and derivatives thereof for their biological activity is well documented and disclosed in, for example, U.S. Pat. Nos. 5,688,486, 5,717,076, 6,452,037, 6,468,244, 6,660,248 and 6,777,445 which teach the use of fullerenes and fullerene derivatives in medical devices, as diagnostic and therapeutic agents and in pharmaceutical compositions for preventing or treating various medical conditions and disorders.

Promising candidates for creation of new bioactive water-soluble fullerene hybrids, which may have desired biological properties, are fullerene-adamantane derivatives, such as the compound suggested by Nakazono M. et al. in Bioorg. Med. Chem. Lett., 2004, 14(22), pp 5619-21. Adamantane (tricyclo[3.3.1.1^{3,7}]decane) is a very stable cycloalkane and the simplest diamondoid which is slightly water-soluble. Amantadine (1-adamantane amine) is an antiviral drug that was approved by the FDA in 1976 for the treatment of influenza type-A in adults and marketed under the brand-name Symmetrel. This drug has also been demonstrated to help reduce symptoms of Parkinson's disease and drug-induced short-term extrapyramidal system syndromes (the extrapyramidal system is a neural network located in the brain that is part of the motor system involved in the coordination of movement). As an antiparkinsonic it is being prescribed together with L-DOPA when L-DOPA responses decline, probably due to tolerance. The mechanism of 1-adamantane amine antiparkinsonic effect is not fully understood, but it appears to be releasing dopamine from the nerve endings of brain cells, together with stimulation of norepinephrine response. The antiviral mechanism of adamantane derivatives, such as amantadine, seems to be unrelated. Amantadine interferes with a viral ion-channel protein M2, which is needed for the viral particle to become “uncoated” once it is taken into the cell by endocytosis. Recently, amantadine was reported to have been used in China poultry farming in an effort to protect the birds against avian flu.

Other adamantyl derivatives have shown excellent efficacy as antiviral, antiglycemic, antiarrhythmic, antidepressant and antitumor agents. Among a broad spectrum of adamantyl-containing therapeutic agents aminoadamantyl derivatives are particularly interesting since they are well-studied compounds that have an extensive array of clinical applications. These applications are ranging from healing of viral infections to treatment of neuroleptic extrapyramidal movement disease, depression and cocaine dependence. Aminoadamantyl derivatives are especially effective in treatment of fatigue associated with multiple sclerosis, Parkinson's and Alzheimer's diseases. On the molecular level, aminoadamantyl derivatives, such as memantine (3,5-dimethyl-adamantan-1-ylamine), were found to function as non-competitive antagonists (channel blockers) for the NMDA receptor. As the latter contributes importantly to the etiology and progression of many neurological diseases states, new aminoadamantyl-fullerene hybrids may have potential to be developed as therapeutic agents for these diseases treatment.

Further use of adamantane and derivatives thereof for their biological activity is well documented and disclosed in, for example, U.S. Pat. Nos. 4,007,181, 4,016,271, 4,061,774, 4,288,609, 5,637,623, 5,880,154, 6,057,364, 6,201,024, 6,214,878, 6,242,470, 6,492,355, 6,720,452, 6,881,754 and 6,927,219 which teach the use of adamantane and adamantyl derivatives as therapeutic agents per se or as part of pharmaceutical compositions for preventing or treating various medical conditions and disorders.

The combination of the therapeutic benefits attainable in fullerenes and adamantane derivatives, put together with the mediator moiety which can improve on the bioavailability while maintaining good biocompatibility of these contributors has yet to be unveiled to date.

There is thus a widely recognized need for, and it would be highly advantageous to have, novel bioavailable and biocompatible fullerene-adamantane hybrid compounds, which could be efficiently utilized as a therapeutic agent in general, and as an antioxidants for the treatment of CNS-associated diseases, disorders and trauma in particular.

### SUMMARY OF THE INVENTION

The present invention is of novel hybrid compounds and uses thereof and, more specifically, to hybrid compounds which comprise a fullerene core attached to one or more glutamate receptor ligand residues via a moiety which renders the compounds aqueous dissolvable (i.e., water soluble) and hence bioavailable under physiological conditions while at the same time capable of crossing the blood-brain barrier. The novel hybrid compounds can therefore serve, inter alia, as antioxidative agents and as therapeutic NMDA antagonists. The present invention is further of methods of preparation of the hybrid compounds and uses thereof as antioxidants and/or neuroprotective agents for the treatment of various medical conditions associated with oxidative stress, neurodegeneration and/or neural damage, as well as other medical conditions as is further delineated herein.

Thus, according to one aspect of the present invention there is provided a compound which includes a fullerene moiety, one or more glutamate receptor ligand residues and one or more bioavailability enhancing moieties and salts, solvates and hydrates thereof.

According to features in preferred embodiments of the invention described below, the bioavailability enhancing moiety includes a backbone of at least 4 atoms. Preferably, the backbone of the bioavailability enhancing moiety includes at least 5 atoms.

According to further features in preferred embodiments of the invention described below, the compound is having sufficient aqueous solubility to render it suitable of being administered in a pharmaceutically effective amount in physiological aqueous media.

According to still further features in the described preferred embodiments the pharmaceutically effective amount ranges from about 10 μg per Kg of body weight to about 600 μg per Kg of body weight per day.

According to still further features in the described preferred embodiments the compound is capable of crossing the blood-brain barrier.

According to still further features in the described preferred embodiments the compound can be represented by a general Formula I:

FX-Z)ₘ Formula I

wherein:

m is an integer of 1-10;

F is the fullerene moiety;

X is the bioavailability enhancing moiety; and

Z is the glutamate receptor ligand residue.

According to still further features in the described preferred embodiments the compound is having a general Formula II:

FMX-Z)_{q})ₘ Formula II

wherein:

M is a first linking moiety; and

q is an integer of 1-10.

According to still further features in the described preferred embodiments the compound is having a general Formula III:

FMX-Y-Z)_{q})ₘ Formula III

wherein:

Y is a second linking moiety.

According to still further features in the described preferred embodiments the bioavailability enhancing moiety has the general formula IV:

-((A)p-D)n- Formula IV

wherein:

p is and integer of 1-10;

n is an integer of 1-100;

A is selected from the group consisting of alkyl, alkenyl, cycloalkyl, cycloalkenyl, heteroalicyclic, aryl and heteroaryl;

D is selected from the group consisting of —O—, —S—, —NRa—, —PRa—, —C(═O)O—, —S(═O)O—, —NRaC(═O)—, —OP(═O)O—, —OS(═O)O— or absent; and

Ra is selected from the group consisting of alkyl and hydroxyl.

According to further features in preferred embodiments of the invention described below, Z is an adamantane residue; X is poly(ethylene glycol); M is a malonic acid residue; Y is C-amide; F is a C60 fullerene moiety; q is 2; and m is 1 or 2. Preferably n is 2-50. More preferably m is 1; and n is 2, 4 or 10 and/or m is 2; and n is 10.

According to yet another aspect of the present invention there is provided a compound having a general Formula V:

MX-Y-Z)_{q} Formula V

wherein:

M is a first linking moiety; and

X is a bioavailability enhancing moiety;

Y is a second linking moiety;

Z is a glutamate receptor ligand residue;

q is an integer of 1-10; and the bioavailability enhancing moiety has the general formula IV. Preferably M is a malonic acid residue; X is poly(ethylene glycol); Z is an adamantane residue; Y is C-amide; A is methylene; p is 2; q is 2; and n is 2, 4 or 10.

According to another aspect of the present invention there is provided a method of synthesizing the compound described below, the method includes:

reacting a bioavailability enhancing moiety and one or more glutamate receptor ligands, to thereby obtain a bioavailability enhancing moiety covalently attached to one or more glutamate receptor ligand residues; and

reacting the bioavailability enhancing moiety covalently attached to the one or more glutamate receptor ligand residues with a fullerene, to thereby obtaining the compound described below.

According to further features in preferred embodiments of the invention described below, the fullerene is covalently attached to the bioavailability enhancing moiety(ies) via a first linking moiety, and the method further includes, prior to reacting the bioavailability enhancing moiety with the glutamate receptor ligand(s):

reacting one or more bioavailability enhancing moiety with a first linking moiety, to thereby obtain one or more bioavailability enhancing moieties covalently attached to the first linking moiety.

According to still further features in the described preferred embodiments the fullerene is covalently attached to one or more bioavailability enhancing moieties via a first linking moiety, and the method further includes, prior to reacting the bioavailability enhancing moiety covalently attached to the one or more glutamate receptor ligand residue with the fullerene:

reacting the bioavailability enhancing moiety covalently attached to the one or more glutamate receptor ligand residues and a first linking moiety, to thereby obtain one or more bioavailability enhancing moieties covalently attached to the glutamate receptor ligand residues at one end and to the first linking moiety at another end.

According to further features in preferred embodiments of the invention described below, the glutamate receptor ligand is attached to the bioavailability enhancing moiety via a second linking moiety.

According to further features in preferred embodiments of the invention described below, the glutamate receptor ligand residue is selected from the group consisting of an N-methyl-D-aspartic acid (NMDA) receptor ligand residue, an (RS)-2-amino-3-(3-hydroxy-5-methyl-4-isoxazolyl)propionic acid (AMPA) receptor ligand residue and a kainic acid (KA) receptor ligand residue.

According to still further features in the described preferred embodiments the glutamate receptor ligand residue is a residue of any of Ligands 1-178 listed in Table A hereinbelow.

According to still further features in the described preferred embodiments the glutamate receptor ligand residue is an N-methyl-D-aspartic acid (NMDA) receptor ligand residue.

According to still further features in the described preferred embodiments the N-methyl-D-aspartic acid (NMDA) receptor ligand residue is an N-methyl-D-aspartic acid (NMDA) receptor antagonist residue.

According to still further features in the described preferred embodiments the N-methyl-D-aspartic acid (NMDA) receptor antagonist residue further includes a cycloalkyl moiety, the cycloalkyl moiety which is selected from the group consisting of an adamantyl, a cubyl, a bicyclo[2.2.1]heptyl, a bicyclo[2.2.2]octyl and a bicyclo[1.1.1]pentyl.

According to still further features in the described preferred embodiments the adamatyl is selected from the group consisting of adamantane residue, memantine residue and amantadine residue.

According to further features in preferred embodiments of the invention described below, the bioavailability enhancing moiety is selected from the group consisting of a poly(alkylene glycol), poly(ethylene imine), poly(vinyl alcohol), poly(methyl vinyl ether), poly(n-isopropyl acrylamide), poly(n,n-dimethyl acrylamide), polyacrylamide and poly(2-hydroxyethyl methacrylate). According to still further features in the described preferred embodiments the poly(alkylene glycol) is selected from the group consisting of poly(ethylene glycol), polypropylene glycol) and poly(butylene glycol). Preferably the poly(alkylene glycol) is poly(ethylene glycol).

According to still further features in the described preferred embodiments the first linking moiety is selected from the group consisting of a malonic acid residue, a 5,6,7,8-tetrahydronaphthalene-diol residue, a 5,6,7,8-tetrahydro-naphthalene-diol residue, a pyrrolidine residue, an aziridine residue and a phosphonate residue. Preferably the first linking moiety is a malonic acid residue.

According to still further features in the described preferred embodiments the second linking moiety is selected from the group consisting of amine, alkyl, alkenyl, cycloalkyl, heteroalicyclic, aryl, heteroaryl, methyleneamine, amine oxide, sulfate, thiosulfate, sulfite, thiosulfite, sulfinate, sulfoxide, sulfonate, S-sulfonamide, N-sulfonamide, disulfide, phosphonate, phosphinyl, phosphine oxide, phosphine sulfide, phosphate, phosphite, thiophosphate, carbonyl, thiocarbonyl, oxime, azo, peroxo, C-carboxylate, O-carboxylate, C-thiocarboxylate, O-thiocarboxylate, N-carbamate, O-carbamate, O-thiocarbamate, N-thiocarbamate, S-dithiocarbamate, N-dithiocarbamate, urea, thiourea, C-amide, N-amide, guanyl, guanidine, hydrazine, hydrazide, thiohydrazide, silyl, siloxy, silaza, silicate, boryl and borate. Preferably the second linking moiety is C-amide.

According to further features in preferred embodiments of the invention described below, the fullerene moiety is selected from the group consisting of a C20 residue, a C24 residue, a C28 residue, a C32 residue, a C34 residue, a C36 residue, a C38 residue, a C40 residue, a C44 residue, a C48 residue, a C50 residue, a C54 residue, a C56 residue, a C60 residue, a C62 residue, a C68 residue, a C70 residue, a C74 residue, a C78 residue, a C80 residue, a C82 residue, a C84 residue, a C86 residue, a C88 residue, a C92 residue, a C94 residue, a C112 residue or a C120 residue. Preferably the fullerene moiety is a C60 residue.

According to an additional aspect of the present invention there is provided a pharmaceutical composition which includes, as an active ingredient, the compound as described herein and a pharmaceutically acceptable carrier.

According to further features in preferred embodiments of the invention described below, the pharmaceutical composition is being packaged in a packaging material and identified in print, in or on the packaging material, for use in the treatment of a medical condition selected from the group consisting of a medical condition in which modulating and/or inhibiting an activity of a glutamate receptor is beneficial, a CNS associated disease, disorder or trauma, an oxidative stress associated disease or disorder, a disease or disorder in which neuroprotection is beneficial, a viral infection, a bacterial infection, cancer and a medical condition at least partially treatable by the compound.

According to yet another aspect of the present invention there is provided a use of the compound presented herein for the treatment of a medical condition selected from the group consisting of a medical condition in which modulating and/or inhibiting an activity of a glutamate receptor is beneficial, a CNS associated disease, disorder or trauma, an oxidative stress associated disease or disorder, a disease or disorder in which neuroprotection is beneficial, a viral infection, a bacterial infection, cancer and a medical condition at least partially treatable by the compound.

According to still another aspect of the present invention there is provided a use of a of the compound presented herein for the preparation of a medicament for the treatment of a medical condition selected from the group consisting of a medical condition in which modulating and/or inhibiting an activity of a glutamate receptor is beneficial, a CNS associated disease, disorder or trauma, an oxidative stress associated disease or disorder, a disease or disorder in which neuroprotection is beneficial, a viral infection, a bacterial infection, cancer and a medical condition at least partially treatable by the compound.

According to yet another aspect of the present invention there is provided a to method of treating a medical condition selected from the group consisting of a medical condition in which modulating and/or inhibiting an activity of a glutamate receptor is beneficial, a CNS associated disease, disorder or trauma, an oxidative stress associated disease or disorder, a disease or disorder in which neuroprotection is beneficial, a viral infection, a bacterial infection, cancer and a medical condition at least partially treatable by the compound of claim 1, the method which includes administering to the subject in need thereof a therapeutically effective amount of the compound.

According to further features in preferred embodiments of the invention described below, the oxidative stress associated disease or disorder is selected from the group consisting of atherosclerosis, an ischemia/reperfusion injury, restenosis, hypertension, cancer, an inflammatory disease or disorder, an acute respiratory distress syndrome (ARDS), asthma, inflammatory bowel disease (IBD), a dermal and/or ocular inflammation, arthritis, metabolic disease or disorder and diabetes.

According to still further features in preferred embodiments the CNS associated disease, disorder or trauma is selected from the group consisting of a neurodegenerative disease or disorder, a stroke, a brain injury and/or trauma, multiple sclerosis, amyotrophic lateral sclerosis, Huntington's disease, Parkinson's disease, Alzheimer's disease, autoimmune encephalomyelitis, AIDS associated dementia, epilepsy, schizophrenia, pain, anxiety, an impairment of memory, a decreased in cognitive and/or intellectual functions, a deterioration of mobility and gait, an altered sleep pattern, a decreased sensory input, a imbalance in the autonomic nerve system, depression, dementia, confusion, catatonia and delirium.

The present invention successfully addresses the shortcomings of the presently known configurations by providing novel hybrid compounds which contain both a fullerene moiety which can exert neuroprotection and/or antioxidant activity, and one or more CNS-active receptor ligand residue attached thereto via one or more bioavailability enhancing moieties which enhances aqueous dissolvability and hence the distribution and delivery of the hybrid compound to and across the blood-brain barrier as well as to other parts of the body.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the singular form “a,” “an,” and “the” include plural references unless the context clearly dictates otherwise. For example, the term “a compound” or “at least one compound” may include a plurality of compounds, including mixtures thereof.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “about” refers to ±10%.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

The term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “active ingredient” refers to a pharmaceutical agent including any natural or synthetic chemical substance that subsequent to its application has, at the very least, at least one desired pharmaceutical or therapeutic effect.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 presents an ESI-MS spectrum of Compound 19, an exemplary hybrid compound according to the present invention wherein the fullerene core is singly substituted with a malonate-bis(adamantyl-polyethyleneglycol) moiety, showing a normal distribution of masses for the final product typical for polyethyleneglycol-derived compounds;

FIG. 2 presents an ESI-MS spectrum of Compound 20, an exemplary hybrid compound according to the present invention wherein the fullerene core is doubly substituted with malonate-bis(adamantyl-polyethyleneglycol) moieties showing a normal distribution of masses for the final product typical for polyethyleneglycol-derived compounds;

FIG. 3 presents a comparative plot showing the effect of treatment with carboxyfullerene, a water soluble derivative of C₆₀ (blue diamonds), Compound 6 (red squares) and Compound 20 (green triangles), two exemplary hybrid compounds according to the present invention, on the mean disease score of four groups of MOG-induced EAE in NOD mice, showing the reduction in severity of the disease in mice treated with Compound 6 and Compound 20 as compared to mice treated with C₆₀ and untreated mice (black squares);

FIG. 4 presents a comparative plot showing the effect of treatment with NBQX (blue diamonds), Compound 20, an exemplary hybrid compound according to the present invention, administered at 30 μg/Kg (green circles) and Compound 20 administered at 300 μg/Kg (red triangles), on the mean disease score of four groups of MOG-induced EAE in NOD mice, showing the reduction in severity of the disease in mice treated with Compound 20 as compared to mice treated with NBQX and untreated mice (black squares);

FIG. 5 presents a series of images of slices the spinal cord of EAE-induced NOD mice after Bielschowsky silver impregnation of axons, showing the reduction of EAE-derived axonal damage in EAE-induced NOD mice as a result of treatment with Compound 20 (3 images on the right) as compared to untreated control mice (4 images on the left), demonstrating the ameliorating effect of an exemplary hybrid compound presented herein in the treatment of EAE; and

FIG. 6 presents a series of images of slices of the spinal cord of EAE-induced NOD mice after staining of axons in the white matter with Luxol fast blue, showing the reduction in demyelination of axons in Compound 20 treated mice (2 images on the right) as compared to untreated control mice (2 images on the left), demonstrating the ameliorating effect of an exemplary hybrid compound presented herein in the treatment of EAE.

### DESCRIPTION OF THE PREFERRED EMBODIMENTS

The present invention is of novel compounds having a beneficial therapeutic activity and uses thereof. More specifically, the present invention is of hybrid compounds which include a fullerene core attached to one or more glutamate receptor ligand residues via a moiety which renders the compounds bioavailable under physiological conditions. The present invention is further of methods of preparation of the hybrid compounds and uses thereof as antioxidants and/or neuroprotective agents for the treatment of medical conditions associated with oxidative stress and/or neural damage, such as, for example, neurological diseases, disorders and trauma, and hence in the treatment of CNS-associated diseases, disorders and trauma, as well as to uses thereof as antiviral, antibacterial, antiglycemic, antiarrhythmic, antidepressant and antitumor agents.

The principles and operation of the present invention may be better understood with reference to the figures and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As is discussed hereinabove, the central nervous system (CNS), governing all function of a living organism, from autonomous functions such as breathing, bowel movements and reflexes to cognitive capacities such as learning, memory and other mental functions, is a highly complex system which is sensitive to any electrical and chemical imbalance. These imbalances are often expressed in what is referred to herein as neurodegenerative diseases and/or CNS-associated diseases, disorders or trauma, causing symptoms which range from mild discomfort to complete impairment and death.

The CNS remains one of the more challenging systems from the therapeutic point of view, especially with respect to the mechanism of action, causes of CNS-associated medical conditions and effective treatments thereof. The advances in CNS research have revealed the important role of neurotransmitters and their receptor targets. Glutamate, one of the main excitatory neurotransmitters in the CNS, is necessary for many normal neurological functions, including learning and memory. Overactivation of glutamate receptors, however, and resulting excitotoxic neuronal injury, has been implicated in the pathogenesis of neuronal loss in the CNS following several acute insults, including hypoxia/ischemia, trauma and certain other neurodegenerative disorders.

Oxidative stress, caused by reactive oxygen species, represents another injury mechanism implicated in many of the same acute and chronic diseases and conditions. Reactive oxygen species, e.g., superoxide radicals, would cause oxidative damage to cellular components, such as peroxidation of cell membrane lipids, inactivation of transport proteins, and inhibition of energy production by mitochondria.

These two events, glutamate excitotoxicity and oxidative stress, may be interlinked; reactive oxygen species formation may occur as a direct consequence of glutamate receptor overstimulation and thus mediate a component of glutamate neurotoxicity. Excitotoxicity, in turn, can be reduced by free radical scavengers, including Cu/Zn-superoxide dismutase and catalase, the 21-aminosteroid “lazaroids”, the vitamin E analog, trolox, spin-trapping agents such as phenylbutyl-N-nitrone, and the ubiquinone analog, idebenone, all reduce the amount of reactive oxygen species.

Free radical scavengers are neuroprotective in cases of traumatic or hypoxic/ischemic CNS injuries while N-methyl-D-aspartate and AMPA/kainate receptor antagonists are neuroprotective in oxygen-glucose deprivation injuries, and reduce loss of brain tissue. Free radical scavengers also protect against excitotoxic neuronal death, and reduce ischemic injury.

Hence, while conceiving the present invention, the inventors have hypothesized that designing compounds which are capable of passing the blood-brain barrier (BBB), and which combine the proven beneficial activity of a radical scavenger together with the proven beneficial activity of glutamate receptor ligands, would result in highly effective neuroprotective agents which will inhibit the progress of a neurodegenerative process by simultaneously acting on both the glutamate excitotoxicity pathway, as well as the oxidative stress pathway. The inventors further conceived that using a fullerene moiety covalently attached to a glutamate receptor ligand residue would serve as an effective, dual action therapeutic agent which can pass the BBB and therefore be capable of treating medical conditions associated with oxidative stress and/or neural damage, such as, for example, neurological diseases, disorders and trauma, and hence for the treatment of CNS-associated diseases, disorders and trauma.

While unsubstituted, pristine fullerenes, as well as several essentially hydrophobic glutamate-receptor ligands and hydrophobic ligands of other receptors are substantially or virtually insoluble in aqueous media, most conjugates thereof are practically insoluble as well. Thus a use thereof as therapeutic agents is impractical due to poor distribution and delivery in the subject. Therefore, while further conceiving the present invention, the inventors hypothesized that introducing highly water soluble moieties into compounds which combine a fullerene moiety and one or more receptor ligand residues, may result in a pharmaceutically viable and novel family of compounds, which are referred to herein hybrid compounds.

While reducing the present invention to practice, the inventors have designed, and successfully prepared and tested family members of this novel family of hybrid compounds, as is further exemplified in the Examples section that follows, which combine all the above desired qualities, namely a radical scavenger in the form of a fullerene moiety, a glutamate anti-excitotoxicity agent in the form of a glutamate receptor ligand residue, both also contribute to the capacity for crossing the BBB, and a bioavailability enhancing moiety which also connects the fullerene moiety and the glutamate receptor ligand residue, and further contributes to the capacity of dissolving in aqueous media.

Thus, according to the present invention there is provided a hybrid compound comprising a fullerene moiety, one or more bioavailability enhancing moieties and one or more glutamate receptor ligand residues.

Preferably, the hybrid compounds described herein do not encompass the compound 61-bis(1-adamantylcarbamoyl)-1,2-methano[60]fullerene.

The terms “moiety” and/or “residue”, as used herein, refer to a major portion of a molecule, which is chemically linked to one or more other molecules.

The phrase “fullerene moiety”, as used herein, refers to a moiety of a compound which is characterized by consisting substantially of carbon and forms a closed spherical structure essentially as presented herein, and having 20, 24, 28, 32, 34, 36, 38, 40, 42, 44, 48, 50, 54, 56, 60, 62, 68, 70, 74, 78, 80, 82, 84, 86, 88, 92, 94, 112 or 120 carbon atoms in all possible arrangements of carbons and in all possible symmetry-related isomers. For additional information regarding nomenclature and classification of fullerenes, see, Cozzi, F., et al., 2005 IUPAC: Pure Appl. Chem., Vol. 77, No. 5, pp. 843-923, 2005. Preferably, the fullerene moiety according to the present invention is a C60 fullerene moiety, consisting of 60 carbon atoms.

The term “bioavailability”, as used herein refers to a degree to which, or a rate at which a drug or other substance is absorbed and distributed in the organism, or becomes available at the site of physiological activity after administration thereof to an organism.

The phrase “bioavailability enhancing moiety”, as used herein refers to a chemical moiety which forms a part of a given compound, and by virtue of its existence as a part of the compound, increases the bioavailability of the compound as compared to a similar compound without this particular moiety.

The phrase “glutamate receptor” refers to all members of a large group of cellular receptors, which include all varieties, forms, splice variants, phases, mutants, subunits and analogs of the ionotropic and the metabotropic glutamate receptor families which include, for example, N-methyl-D-aspartic acid (NMDA) receptor, the (RS)-2-amino-3-(3-hydroxy-5-methyl-4-isoxazolyl)propionic acid (AMPA) receptor and the kainic acid (KA) receptor. The glutamate receptors are multimeric assemblies of four or five subunits, which play a vital role in the mediation of excitatory synaptic transmission. This process is the means by which cells in the brain (neurons) communicate with each other. The receptors themselves are ligand gated ion channels, i.e., upon binding of glutamate that has been released from a companion cell, charged ions such as Na+ and Ca2+ pass through a channel in the centre of the receptor complex. This flow of ions results in a depolarisation of the plasma membrane and the generation of an electrical current that is propagated down the processes (dendrites and axons) of the neuron to the next in line.

The phrase “receptor ligand”, as used herein refers to a small molecule that binds to a site on a macromolecule's surface by intermolecular forces. This binding is usually reversible as actual coordinate covalent bonds between a ligand and the macromolecule are rare in biological systems. Ligand binding typically leads to a structural rearrangement in or of the macromolecules, therefore altering their susceptibility to participating in other ligands and/or types of chemical reactions. Thus, a substrate is a specific case of a ligand that in subsequent reactions is transformed into another chemical compound, or product. Other types of ligands include inhibitors, activators, agonist, antagonists and neurotransmitters, all of which are of several types.

The term “effector” is also commonly used which includes all of the abovementioned ligands, and therefore interchangeable with the term ligand.

The phrase “receptor ligand”, according to the present invention, encompasses naturally occurring ligands as well as analogs, derivatives, structural mimics and biological activity mimics thereof.

As fullerene derivatives as well as some receptor ligand residues are known to be effective and pharmaceutically viable therapeutic agents, the bioavailability enhancing moiety and its metabolic break-down products, according to the present invention, are selected such that they are also pharmaceutically viable in mammals.

Thus, the main purpose of having a bioavailability enhancing moiety is to enable the crossing of the hybrid compound to the brain, through the BBB via bodily circulation systems such as the blood system, and therefore the main contribution of the bioavailability enhancing moiety is to improve the aqueous solubility of the hybrid compounds presented herein as compared to compounds which do not include such moieties. The aqueous solubility of the hybrid compounds is required to be high enough so as to allow the hybrid compounds to interact with their target(s) such as, for example, an enzyme, a receptor, an adduct counterpart and another chemical species, and exert an impact thereon such as, for example, inhibition of, excitation of, activation of, conformational change of, binding with, reacting with, blocking of, hybridizing with, exchanging with and displacing its target. Hence, the bioavailability enhancing moiety increases the aqueous solubility of the hybrid compound so as to allow the hybrid compounds presented herein to sufficiently dissolve in physiological aqueous media so as to be effectively administered in a pharmaceutically effective amount, as this phrase is defined hereinbelow, and efficiently circulate in the body.

In a preferred embodiment of the present invention, a bioavailability enhancing moiety comprises at least four atoms in its backbone chain, and preferably at least 5, 6, 7 or more atom-long backbone chain, preferably interrupted and/or substituted by one or more heteroatoms and/or other polarizable chemical groups and substituents such as, for example, H-bond forming elements, non-bonding electron-pair containing elements, aromatic moieties which comprise pi systems, electron-withdrawing/pushing substituents and partially ionizable moieties, as is further defined, exemplified and discussed in detail hereinbelow.

The phrase “physiological aqueous media”, as used herein refers to the main physiological carrier media of a mammal which are essentially aqueous media such as, for example, the blood, the lymph plasma, the cerebro-spinal fluid (CSF), the extracellular media and the intracellular cytoplasm.

In the context of the present invention, an effective concentration in physiological aqueous media relates to the phrase “therapeutically effective amount”, as this is defined hereinbelow, in that the attainable concentration of the hybrid compounds allows the hybrid compounds presented herein to be administered to a subject as therapeutic agents by conventional methods at a therapeutically effective amount thereof as needed to impart a therapeutic effect on the subject.

The hybrid compounds of the present invention are designed such that they reach an effective concentration in physiological aqueous media, as demonstrated and exemplified in the Examples section that follows, wherein an exemplary hybrid compound was dissolved at a concentration of 51.5×10⁻⁵ M in an aqueous media containing 2% DMSO.

Solubility, as this term is used in the context of the present invention, is the maximum amount of a solute that dissolves in a given quantity of solvent at a specific temperature and pressure. Common measures of solubility include the mass of solute per unit mass of solution (mass fraction), mole fraction of solute, molality, molarity, and others.

According to preferred embodiments of the present invention, the compounds presented herein are characterized by an aqueous solubility in water containing 2% DMSO which is equal or greater than 0.00001 M (10 μM), as determined by conventional methods at standard temperature and pressure conditions (STP). Preferably, the maximal aqueous solubility of the compounds of the present invention is equal or greater than 0.00005 M (50 μM), more preferably equals or greater than 0.0001 M (100 more preferably equals or greater than 0.0005 M (500 μM), more preferably equals or greater than 0.001 M (1.0 mM), more preferably equals or greater than 0.005 M (5.0 mM) and more preferably equals or greater than 0.01 M (10 mM).

The bioavailability enhancing moiety is further selected such that it improves the aqueous solubility of the hybrid compounds while not harming the capacity of the hybrid compound to cross the BBB, hence be, for example, amphiphilic and uncharged. General and specific examples of bioavailability enhancing moieties are presented hereinbelow.

The hybrid compound according to the present invention can therefore be represented by the general Formula I below:

FX-Z)ₘ Formula I

wherein:

F is a fullerene moiety;

X is a bioavailability enhancing moiety;

Z is a glutamate receptor ligand residue; and

m is an integer representing the number of bioavailability enhancing moieties attached to the fullerene moiety, each carrying a glutamate receptor ligand residue; and whereas:

m ranges from 1 to 10. Preferably, m ranges from 1 to 4 and more preferably m ranges from 1 to 2. An example of a hybrid compound wherein m is 2 is presented in Compound 20 in the Example section that follows.

The bioavailability enhancing moiety can be directly attached to the fullerene moiety directly or via a linking moiety, referred to herein as the first linking moiety. There may be more than one such first linking moieties attached to the fullerene moiety, and each of these first linking moieties can be attached to more than one bioavailability enhancing moieties. Hence, according to preferred embodiments of the invention, the hybrid compounds presented herein can be represented by the general Formula II below:

FMX-Z)_{q})ₘ Formula II

wherein:

M is a first linking moiety; and

q is an integer representing the number of linking moieties attached to the fullerene moiety, each is attached to more than one bioavailability enhancing moiety, which in turn is attached to a glutamate receptor ligand residue; and whereas q ranges from 1 to 10. Preferably, q ranges from 1 to 4 and more preferably q ranges from 1 to 2. Example of hybrid compounds wherein q is 2 are presented in Compounds 6, 12, 16, 19 and 20 in the Example section that follows.

The glutamate receptor ligand residue can be attached to the bioavailability enhancing moiety directly or via another linking moiety which is referred to herein as the second linking moiety, hence, according to preferred embodiments, the hybrid compounds presented herein can be represented by the general Formula III below:

FMX-Y-Z)_{q})ₘ Formula III

wherein Y is a second linking moiety.

As discussed hereinabove, the bioavailability enhancing moiety is selected or prepared so as to render the hybrid compound sufficiently aqueous soluble, while maintaining it capacity to cross the BBB. To that end, the bioavailability enhancing moiety, denoted X in Formulae I, II and III, is required to exhibit a balance between polarity and hydrophobicity, by including polarizable groups such as, for example heteroatoms, and hydrophobic groups such as, for example, hydrocarbon groups, and be essentially neutral. Hence, according to preferred embodiments of the present invention, the bioavailability enhancing moiety denoted X in Formulae I, II and III, can be represented by the general Formula IV:

-((A)p-D)n- Formula IV

wherein:

p and n are each independently an integer;

A is selected from the group consisting of alkyl, alkenyl, cycloalkyl, cycloalkenyl, heteroalicyclic, aryl and heteroaryl;

D is selected from the group consisting of —O—, —S—, —NRa—, —PRa—, —C(═O)O—, —S(═O)O—, —NRaC(═O)—, —OP(═O)O—, —OS(═O)O— or absent;

and whereas p ranges from 1 to 10, n ranges from 1 to 100 and Ra is selected from the group consisting of alkyl and hydroxyl. Preferably, A is a methylene group, p ranges from 2 to 4 and n ranges from 2 to 50, and more preferably p is 2 and n ranges from 2 to 10. Example of these preferred hybrid compounds wherein A is a methylene group, p is 2 and n ranges from 2 to 10 are presented in Compounds 6, 16, 19 and 20 in the Example section that follows.

As discussed hereinabove, the hybrid compounds of the present invention are preferably directed at exerting a therapeutic effect in the CNS. Therefore, the glutamate receptor ligand residue which forms a part of the hybrid compound is selected so as to interact with crucial components of the CNS such as the various receptors which are regulated by various ligands and neurotransmitters in the CNS. Preferably the interaction is a specific interaction, targeting a specific receptor, by using a receptor-specific ligand thereof. Such receptors may include other receptors than the glutamate receptors family, such as, for example, gamma amino butyric acid (GABA) receptors family, glycine receptors family, aspartic acid (aspartate) receptors family, acetylcholine receptors family, dopamine receptors family, norepinephrine (noradrenalin) receptors family, serotonin (5-hydroxytryptamine, 5-ht) receptors family and receptors of other neurotransmitters and excitatory/inhibitory amino acids, derivatives, analogs and oligomers thereof.

According to preferred embodiments, the hybrid compounds of the present invention includes a residue of ligand which is specific to the glutamate (Glu) receptors family, which includes, for example, the N-methyl-D-aspartic acid (NMDA) receptors family, the (RS)-2-amino-3-(3-hydroxy-5-methyl-4-isoxazolyl)propionic acid (AMPA) receptors family and the kainic acid (KA) receptors family. More preferably, the ligand residue is an NMDA receptor ligand residue, and most preferably the NMDA receptor ligand residue is an antagonist thereof.

The literature is endowed with a myriad of publications pertaining to compounds which exhibit glutamate receptor modulation activity, i.e., acting as ligands thereof. For a non-limiting example, one group of researchers which presented the results of their search after NMDA receptor antagonist in U.S. patent application having the publication No. 20050032881 described the NMDA receptor antagonist residue in the most general way, and thus U.S. Patent Application having the publication No. 20050032881 is incorporated herein in its entirety.

Another publication which reviews and discuss a large group of ligands for the glutamate receptors family, was presented by Hans Brauner-Osborne et al. in J. Med. Chem., 2000, Vol. 43, No. 14, and is therefore also incorporated herein in its entirety. The ligands which are discussed and presented in the publication by Bräuner-Osborne et al. are set forth in Table A hereinbelow.

TABLE A
Structures of exemplary naturally occurring amino acids showing effects on Glu receptors

| | |
|---|---|
| Ligand 1 | 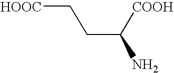 |
| Ligand 2 | 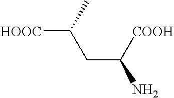 |
| Ligand 3 | 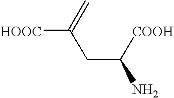 |
| Ligand 4 | 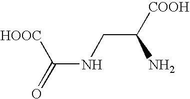 |
| Ligand 5 | 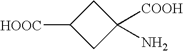 |
| Ligand 6 | 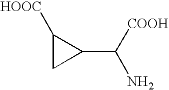 |
| Ligand 7 | 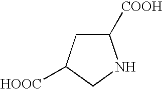 |
| Ligand 8 | 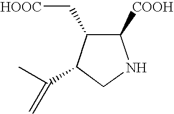 |
| Ligand 9 | 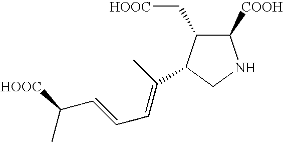 |
| Ligand 10 | 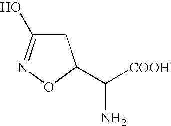 |
| Ligand 11 | 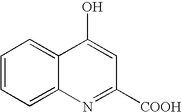 |
| Ligand 12 | 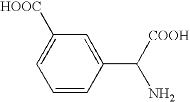 |
| Ligand 13 | 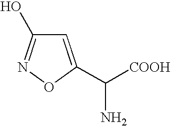 |
| Ligand 14 | 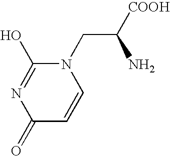 |
| Ligand 15 | 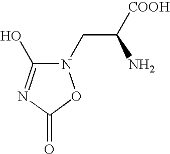 |

Schematic illustration of the multiplicity of excitatory amino acids receptors and the structures of exemplary key ligands

| | |
|---|---|
| Ligand 16 | 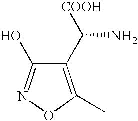 |
| Ligand 17 | 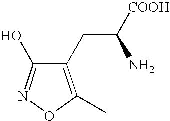 |
| Ligand 18 | 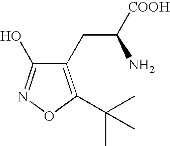 |
| Ligand 19 | 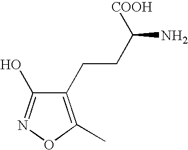 |
| Ligand 20 | 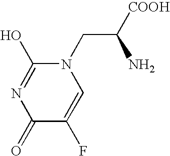 |
| Ligand 21 | 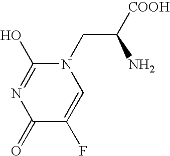 |
| Ligand 22 | 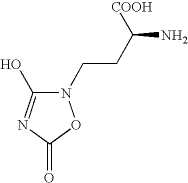 |

Structures of N-methyl-D-aspartic acid (NMDA) and exemplary NMDA receptor agonists

| | |
|---|---|
| Ligand 23 | 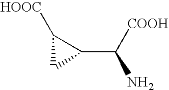 |
| Ligand 24 | 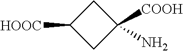 |
| Ligand 25 | 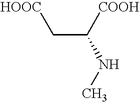 |
| Ligand 26 | 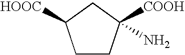 |
| Ligand 27 | 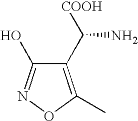 |
| Ligand 28 | 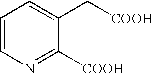 |
| Ligand 29 | 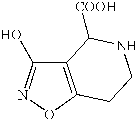 |
| Ligand 30 | 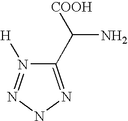 |

Structures of exemplary competitive NMDA receptor antagonists

| | |
|---|---|
| Ligand 31 | 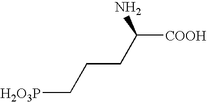 |
| Ligand 32 | 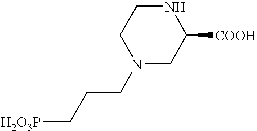 |
| Ligand 33 | 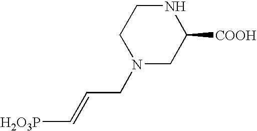 |
| Ligand 34 | 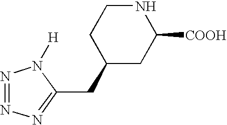 |
| Ligand 35 | 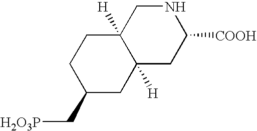 |
| Ligand 36 | 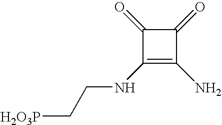 |

Structures of exemplary noncompetitive NMDA receptor antagonists

| | |
|---|---|
| Ligand 37 | 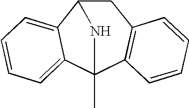 |
| Ligand 38 | 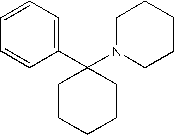 |
| Ligand 39 | 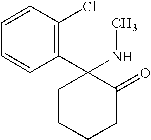 |
| Ligand 49 | 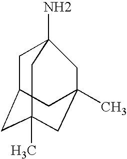 |
| Ligand 41 | 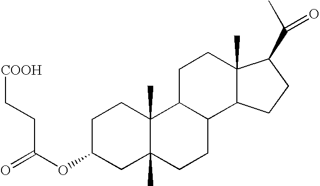 |
| Ligand 42 | 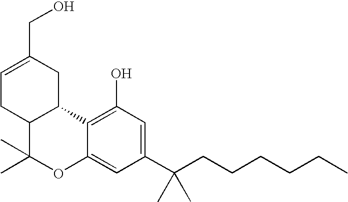 |
| Ligand 43 | 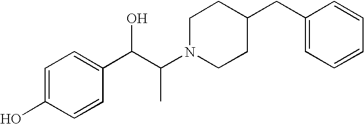 |
| Ligand 44 | 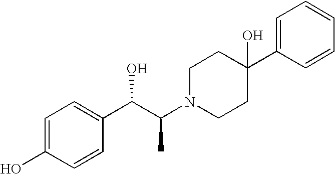 |

Structures of exemplary compounds showing agonist or partial agonist effects at the co-transmitter glycine site
(GlycineB receptor) of the NMDA receptor complex

| | |
|---|---|
| Ligand 45 | 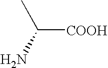 |
| Ligand 46 | 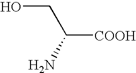 |
| Ligand 47 | 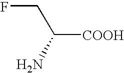 |
| Ligand 48 | 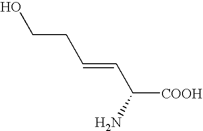 |
| Ligand 49 | 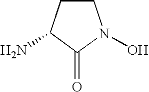 |
| Ligand 50 | 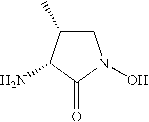 |
| Ligand 51 | 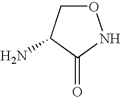 |
| Ligand 52 | 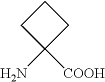 |

Structures of exemplary compounds showing antagonist effects at the co-transmitter glycine site
(GlycineB receptor) of the NMDA receptor complex

| | |
|---|---|
| Ligand 53 | 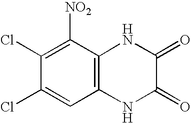 |
| Ligand 54 | 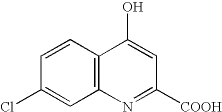 |
| Ligand 55 | 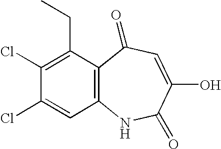 |
| Ligand 56 | 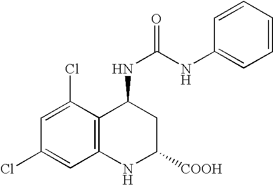 |
| Ligand 57 | 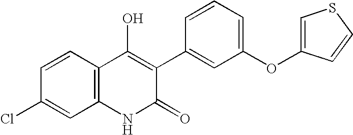 |
| Ligand 58 | 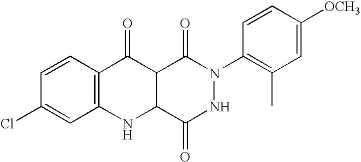 |
| Ligand 59 | 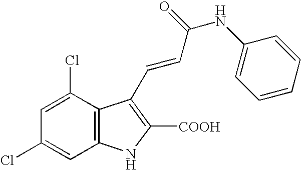 |

Structures of exemplary AMPA receptor agonists

| | |
|---|---|
| Ligand 60 | 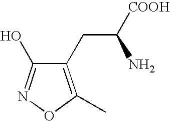 |
| Ligand 61 | 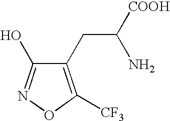 |
| Ligand 62 | 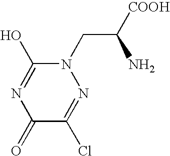 |
| Ligand 63 | 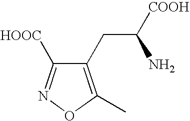 |
| Ligand 64 | 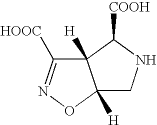 |
| Ligand 65 | 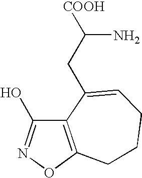 |
| Ligand 66 | 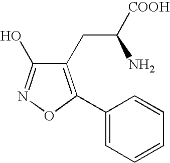 |
| Ligand 67 | 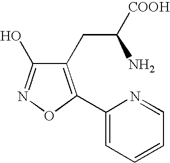 |
| Ligand 68 | 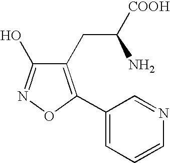 |
| Ligand 69 | 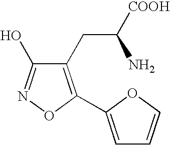 |
| Ligand 70 | 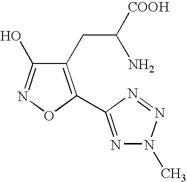 |
| Ligand 71 | 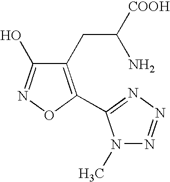 |

Structures of exemplary competitive AMPA receptor antagonists

| | |
|---|---|
| Ligand 72 | 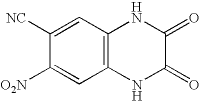 |
| Ligand 73 | 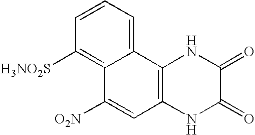 |
| Ligand 74 | 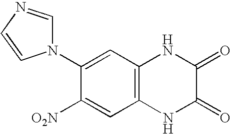 |
| Ligand 75 | 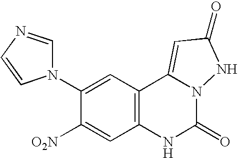 |
| Ligand 76 | 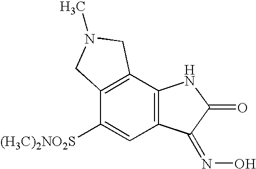 |
| Ligand 77 | 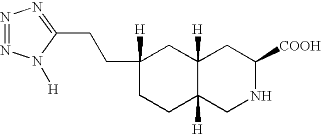 |
| Ligand 78 | 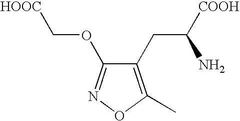 |
| Ligand 79 | 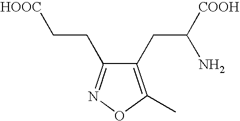 |
| Ligand 80 | 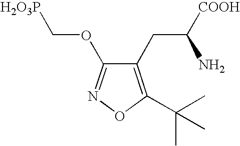 |
| Ligand 81 | 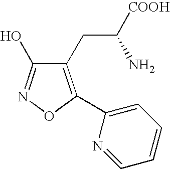 |
| Ligand 82 | 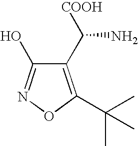 |

Structures of exemplary noncompetitive AMPA receptor antagonists (Ligands 83-86) and some AMPA receptor
modulatory agents (“Ampakines”) (Ligands 87-90)

| | |
|---|---|
| Ligand 83 | 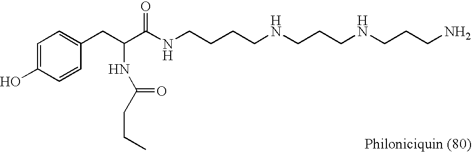 |
| Ligand 84 | 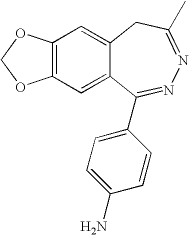 |
| Ligand 85 | 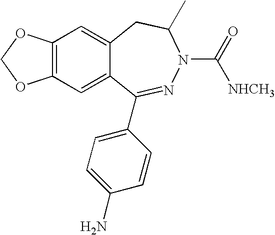 |
| Ligand 86 | 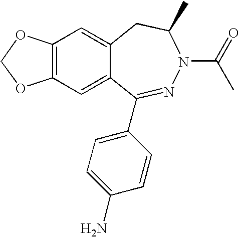 |
| Ligand 87 | 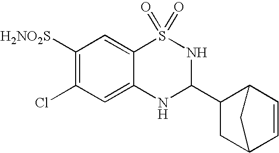 |
| Ligand 88 | 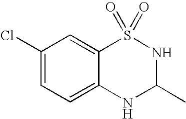 |
| Ligand 89 | 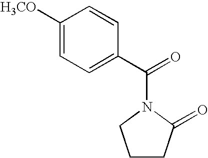 |
| Ligand 90 | 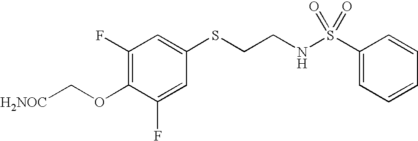 |

Structures of exemplary Kainate receptor agonists

| | |
|---|---|
| Ligand 91 | 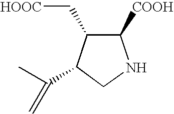 |
| Ligand 92 | 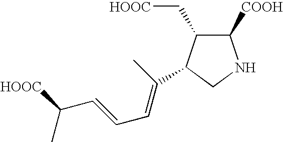 |
| Ligand 93 | 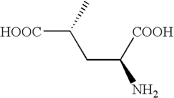 |
| Ligand 94 | 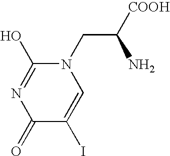 |
| Ligand 95 | 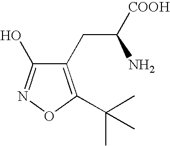 |
| Ligand 96 | 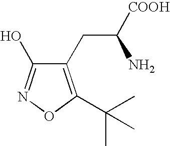 |
| Ligand 97 | 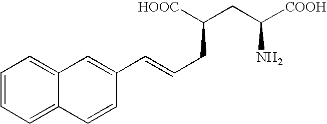 |

Structures of exemplary competitive KA receptor antagonists

| | |
|---|---|
| Ligand 98 | 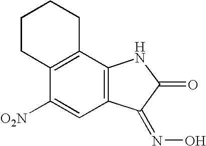 |
| Ligand 99 | 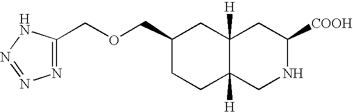 |
| Ligand 100 | 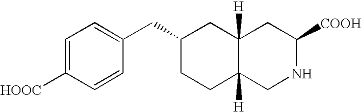 |

Structures of exemplary amino acids showing agonist effects at metabotropic Glu receptors (mGluRs)

| | | |
|---|---|---|
| Ligand 101 | 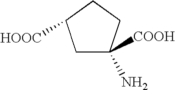 | |
| Ligand 102 Ligand 103 Ligand 104 | 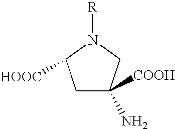 | 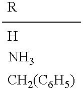 |
| Ligand 105 | 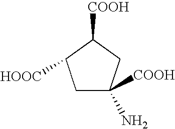 | |
| Ligand 106 | 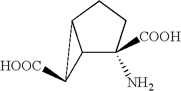 | |
| Ligand 107 | 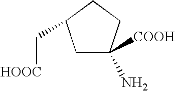 | |
| Ligand 108 | 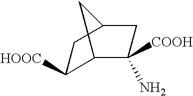 | |
| Ligand 109 | 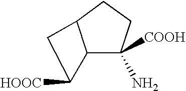 | |
| Ligand 110 | 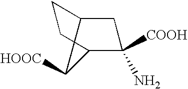 | |
| Ligand 111 Ligand 112 Ligand 113 | 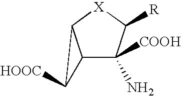 | 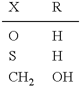 |

Structures of exemplary Glu analogues showing effects at ionotropic Glu receptors (iGluRs) and/or metabotropic Glu receptors
(mGluRs) (middle column) and the corresponding homologues interacting preferentially with mGluRs (right column)

| | | |
|---|---|---|
| Ligand 115, Ligand 114 | 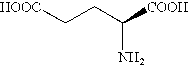 | 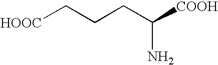 |
| Ligand 117, Ligand 116 | 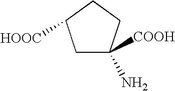 | 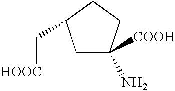 |
| Ligand 119, Ligand 118 | 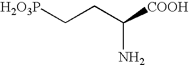 | 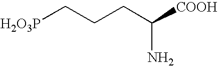 |
| Ligand 121, Ligand 120 | 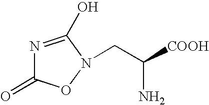 | 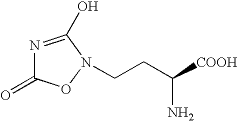 |
| Ligand 123, Ligand 122 | 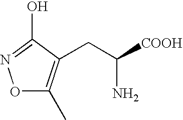 | 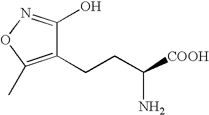 |
| Ligand 125, Ligand 124 Ligand 125 Ligand 126 | 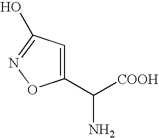 | 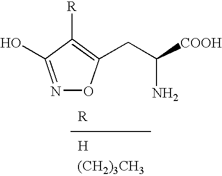 |

Structures of exemplary amino acids showing competitive antagonist effects at metabotropic Glu receptors

| | | |
|---|---|---|
| Ligand 128 Ligand 129 Ligand 130 | 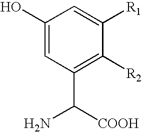 | 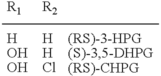 |
| Ligand 131 Ligand 132 | 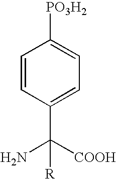 | 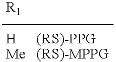 |
| Ligand 133 | 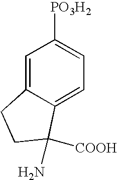 | |
| Ligand 134 Ligand 135 Ligand 136 Ligand 137 Ligand 138 Ligand 139 | 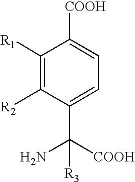 | 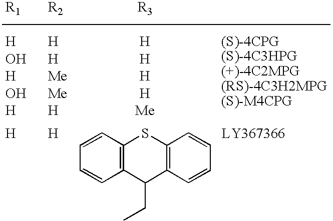 |
| Ligand 140 | 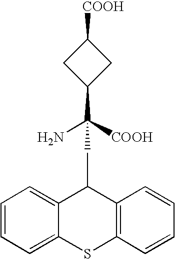 | |
| Ligand 141 | 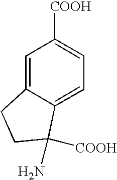 | |
| Ligand 142 | 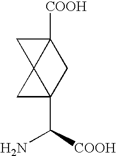 | |
| Ligand 143 | 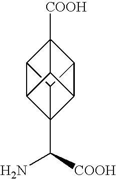 | |

Structures of some amino acids showing agonist or competitive antagonist effects at metabotropic Glu receptors

| | | |
|---|---|---|
| Ligand 143 Ligand 144 | 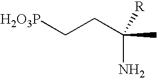 | 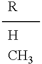 |
| Ligand 145 Ligand 146 Ligand 147 Ligand 148 | 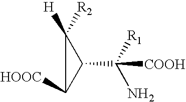 | 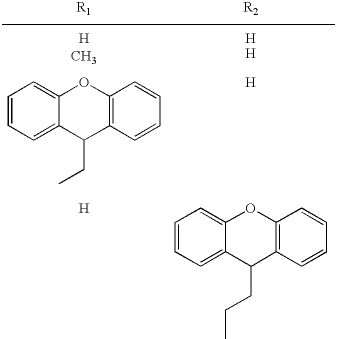 |
| Ligand 149 Ligand 150 | 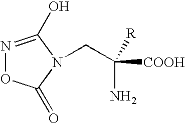 | 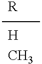 |
| Ligand 151 | 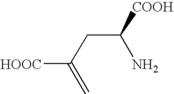 | |
| Ligand 152 Ligand 153 Ligand 154 | 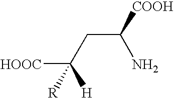 | 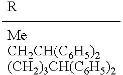 |

Structures of exemplary compounds showing noncompetitive antagonist effects at metabotropic Glu receptors

| | | |
|---|---|---|
| Ligand 155 | 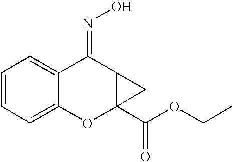 | |
| Ligand 156 | 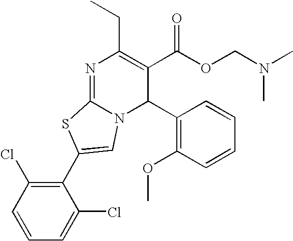 | |
| Ligand 157 | 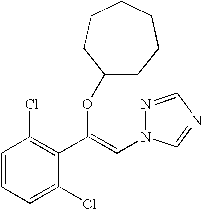 | |
| Ligand 158 Ligand 159 | 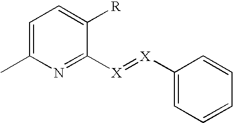 | 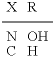 |
| Ligand 160 | 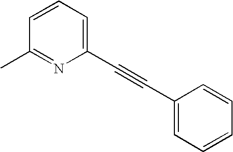 | |

Structures of exemplary jonotropic Glu receptor antagonists of clinical interest

| | | |
|---|---|---|
| Ligand 161 | 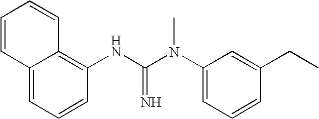 | |
| Ligand 162 | 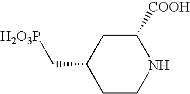 | |
| Ligand 163 | 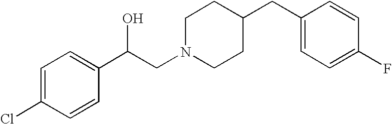 | |
| Ligand 164 Ligand 165 | 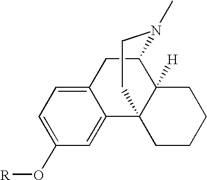 | R = H R = CH₃ |
| Ligand 166 | 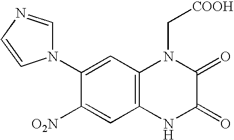 | |
| Ligand 168 | 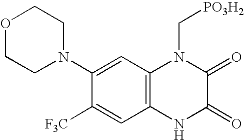 | |
| Ligand 169 | 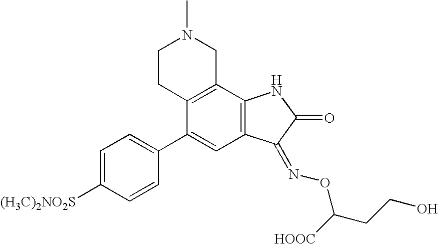 | |

Structures of exemplary ionotropic Glu receptor ligands or modulatory agents of interest as experimental tools or therapeutic agents

| | |
|---|---|
| Ligand 170 | 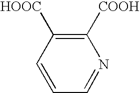 |
| Ligand 171 | 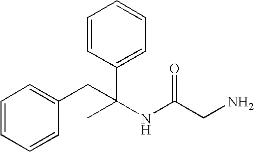 |
| Ligand 172 | 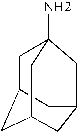 |
| Ligand 173 | 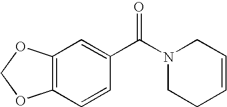 |
| Ligand 174 | 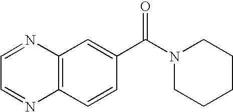 |

Exemplary ionotropic Glu receptor antagonists of therapeutic interest

| | |
|---|---|
| Ligand 175 | 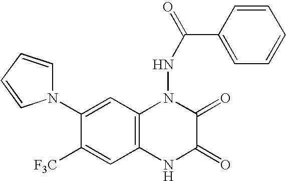 |
| Ligand 176 | 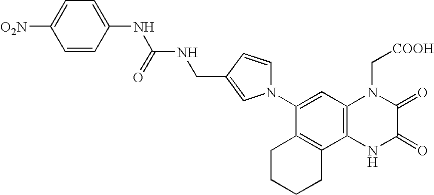 |
| Ligand 177 | 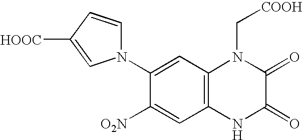 |
| Ligand 178 | 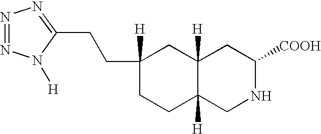 |

Hence, according to embodiments of the present invention, the glutamate receptor ligand residue is a residue of any of Ligands 1-178 listed in Table A hereinabove and functional and structural mimetics thereof.

According to preferred embodiments of the present invention, the glutamate receptor ligand residue is an N-methyl-D-aspartic acid (NMDA) receptor ligand residue (see, Ligands 23-59 in Table A), and more preferably the NMDA receptor ligand residue is a residue of an NMDA receptor antagonist (see, Ligands 31-44 and 53-59 in Table A).

Preferably, the NMDA receptor antagonist residue, according to preferred embodiments of the invention, is a cycloalkyl moiety selected from the group consisting of an adamantyl, a cubyl, a bicyclo[2.2.1]heptyl, a bicyclo[2.2.2]octyl and a bicyclo[1.1.1]pentyl, optionally further substituted by one substituent or more, selected from the group consisting of amine, alkyl, alkenyl, cycloalkyl, heteroalicyclic, aryl, heteroaryl, methyleneamine, amine oxide, sulfate, thiosulfate, sulfite, thiosulfite, sulfinate, sulfoxide, sulfonate, S-sulfonamide, N-sulfonamide, disulfide, phosphonate, phosphinyl, phosphine oxide, phosphine sulfide, phosphate, phosphite, thiophosphate, carbonyl, thiocarbonyl, oxime, azo, peroxo, C-carboxylate, O-carboxylate, C-thiocarboxylate, O-thiocarboxylate, N-carbamate, O-carbamate, O-thiocarbamate, N-thiocarbamate, S-dithiocarbamate, N-dithiocarbamate, urea, thiourea, C-amide, N-amide, guanyl, guanidine, hydrazine, hydrazide, thiohydrazide, silyl, siloxy, silaza, silicate, boryl and borate.

More preferably, the NMDA receptor antagonist residue, according to the present invention, is an adamatyl residue which is selected from the group consisting of adamantane residue, memantine (3,5-dimethyl-adamantan-1-ylamine, see, Ligand 40 in Table A) residue and amantadine (adamantan-1-ylamine, see, Ligand 172 in Table A) residue.

The term “amine” is used herein to describe a NR′R″ group in cases where the amine is an end group, as defined hereunder, and is used herein to describe a —NR′— group in cases where the amine is a linking group.

Herein throughout, the phrase “linking moiety” describes a group (a substituent) that is attached to another moiety in the compound via two or more atoms thereof. In order to differentiate a linking group from a substituent that is attached to another moiety in the compound via one atom thereof, the latter will be referred to herein and throughout as an “end group”.

The term “alkyl” describes a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be interrupted by 1-3 heteroatoms, such as, for example, O, N, S and/or P. The alkyl group may be substituted or unsubstituted. Substituted alkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

The alkyl group can be an end group, as this phrase is defined hereinabove, wherein it is attached to a single adjacent atom, or a linking moiety, as this phrase is defined hereinabove, which connects two or more moieties via at least two carbons in its chain.

The term “cycloalkyl” describes an all-carbon monocyclic, polycyclic or fused ring (i.e., rings which share an adjacent pair of carbon atoms) group where one or more of the rings does not have a completely conjugated pi-electron system. Non-limiting examples of cycloalkyl according to the present invention, include adamantyl, cubyl, bicyclo[2.2.1]heptyl, bicyclo[2.2.2]octyl and a bicyclo[1.1.1]pentyl. The cycloalkyl group may be substituted or unsubstituted. Substituted cycloalkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The cycloalkyl group can be an end group, as this phrase is defined hereinabove, wherein it is attached to a single adjacent atom, or a linking moiety, as this phrase is defined hereinabove, connecting two or more moieties at two or more positions thereof.

The term “aryl” describes an all-carbon monocyclic or fused-ring polycyclic (i.e., rings which share adjacent pairs of carbon atoms, also referred to as polyaryls) groups having a completely conjugated pi-electron system. Non-limiting examples of aryls include benzene (phenyl), pentalene, indene, naphthalene, anthracene, pyrene, triphenylene, phenalene and coronene. The aryl group may be substituted or unsubstituted. Substituted aryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The aryl group can be an end group, as this term is defined hereinabove, wherein it is attached to a single adjacent atom, or a linking moiety, as this term is defined hereinabove, connecting two or more moieties at two or more positions thereof.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. Substituted heteroaryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroaryl group can be an end group, as this phrase is defined hereinabove, where it is attached to a single adjacent atom, or a linking moiety, as this phrase is defined hereinabove, connecting two or more moieties at two or more positions thereof.

The term “heteroalicyclic” describes a monocyclic, polycyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. Substituted heteroalicyclic—may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroalicyclic group can be an end group, as this phrase is defined hereinabove, where it is attached to a single adjacent atom, or a linking moiety, as this phrase is defined hereinabove, connecting two or more moieties at two or more positions thereof. Representative examples are piperidine, piperazine, tetrahydrofurane, tetrahydropyrane, morpholino and the like.

The term “amine-oxide” describes a —N(OR′)(R″) or a —N(OR′)— group, where R′ and R″ are as defined herein. This term refers to a —N(OR′)(R″) group in cases where the amine-oxide is an end group, as this phrase is defined hereinabove, and to a —N(OR′)— group in cases where the amine-oxime is a linking moiety, as this phrase is defined hereinabove.

The term “halide” and “halo” describes fluorine, chlorine, bromine or iodine.

The term “haloalkyl” describes an alkyl group as defined above, further substituted by one or more halide.

The term “sulfate” describes a —O—S(═O)₂—OR′ end group, as this term is defined hereinabove, or an —O—S(═O)₂—O— linking moiety, as these phrases are defined hereinabove, where R′ is as defined hereinabove.

The term “thiosulfate” describes a —O—S(═S)(═O)—OR′ end group or a —O—S(═S)(═O)—O— linking moiety, as these phrases are defined hereinabove, where R′ is as defined hereinabove.

The term “sulfite” describes an —O—S(═O)—O—R′ end group or a —O—S(═O)— group linking moiety, as these phrases are defined hereinabove, where R′ is as defined hereinabove.

The term “thiosulfite” describes a —O—S(═S)—O—R′ end group or an —O—S(═S)—O— group linking moiety, as these phrases are defined hereinabove, where R′ is as defined hereinabove.

The term “sulfinate” describes a —S(═O)—OR′ end group or an —S(═O)— group linking moiety, as these phrases are defined hereinabove, where R′ is as defined hereinabove.

The term “sulfoxide” or “sulfinyl” describes a —S(═O)R′ end group or an —S(═O)— linking moiety, as these phrases are defined hereinabove, where R′ is as defined hereinabove.

The term “sulfonate” describes a —S(═O)₂—R′ end group or an —S(═O)₂— linking moiety, as these phrases are defined hereinabove, where R′ is as defined herein.

The term “S-sulfonamide” describes a —S(═O)₂—NR′R″ end group or a —S(═O)₂—NR′— linking moiety, as these phrases are defined hereinabove, with R′ and R″ as defined herein.

The term “N-sulfonamide” describes an R′S(═O)₂—NR″— end group or a —S(═O)₂—NR′— linking moiety, as these phrases are defined hereinabove, where R′ and R″ are as defined herein.

The term “disulfide” refers to a —S—SR′ end group or a —S—S— linking moiety, as these phrases are defined hereinabove, where R′ is as defined herein.

The term “phosphonate” describes a —P(═O)(OR′)(OR″) end group or a —P(═O)(OR′)(O)— linking moiety, as these phrases are defined hereinabove, with R′ and R″ as defined herein.

The term “phosphinyl” describes a PR′R″ end group or a —PR′— linking moiety, as these phrases are defined hereinabove, with R′ and R″ as defined hereinabove.

The term “phosphine oxide” describes a —P(═O)(R′)(R″) end group or a —P(═O)(R′)— linking moiety, as these phrases are defined hereinabove, with R′ and R″ as defined herein.

The term “phosphine sulfide” describes a P(═S)(R′)(R″) end group or a —P(═S)(R′)— linking moiety, as these phrases are defined hereinabove, with R′ and R″ as defined herein.

The term “phosphate” describes an —O—P(═O)(OR′)(OR″) end group or an —O—P(═O)(OR′)(O)— linking moiety, as these phrases are defined hereinabove, with R′, R″ as defined herein.

The term “phosphite” describes an —O—PR′(═O)(OR″) end group or an —O—PR′(═O)(O)— linking moiety, as these phrases are defined hereinabove, with R′ and R″ as defined herein.

The term “thiophosphate” describes an —O—P(═S)(OR′)(OR″) end group or an —O—P(═S)(OR')(O)— linking moiety, as these phrases are defined hereinabove, with R′, R″ as defined herein.

The term “carbonyl” or “carbonate” as used herein, describes a —C(═O)—R′ end group or a —C(═O)— linking moiety, as these phrases are defined hereinabove, with R′ as defined herein.

The term “thiocarbonyl” as used herein, describes a —C(═S)—R′ end group or a —C(═S)— linking moiety, as these phrases are defined hereinabove, with R′ as defined herein.

The term “oxime” describes a ═N—OH end group or a ═N—O— linking moiety, as these phrases are defined hereinabove.

The term “hydroxyl” describes a —OH group.

The term “alkoxy” describes both an —O-alkyl and an —O-cycloalkyl group, as defined herein.

The term “aryloxy” describes both an —O-aryl and an —O-heteroaryl group, as defined herein.

The term “thiohydroxy” describes a —SH group.

The term “thioalkoxy” describes both a —S-alkyl group, and a —S-cycloalkyl group, as defined herein.

The term “thioaryloxy” describes both a —S-aryl and a —S-heteroaryl group, as defined herein.

The term “cyano” describes a —C≡N group.

The term “isocyanate” describes an N═C═O group.

The term “nitro” describes an —NO₂ group.

The term “acyl halide” describes a —(C═O)Rx group wherein Rx is halide, as defined hereinabove.

The term “azo” or “diazo” describes an —N═NR′ end group or an —N═N— linking moiety, as these phrases are defined hereinabove, with R′ as defined hereinabove.

The term “peroxo” describes an —O—OR′ end group or an —O—O— linking moiety, as these phrases are defined hereinabove, with R′ as defined hereinabove.

The term “C-carboxylate” describes a —C(═O)—OR′ end group or a —C(═O)— linking moiety, as these phrases are defined hereinabove, where R′ is as defined herein.

The term “O-carboxylate” describes a —OC(═O)R′ end group or a —OC(═O)— linking moiety, as these phrases are defined hereinabove, where R′ is as defined herein.

The term “C-thiocarboxylate” describes a —C(═S)—OR′ end group or a —C(═S)—O— linking moiety, as these phrases are defined hereinabove, where R′ is as defined herein.

The term “O-thiocarboxylate” describes a —OC(═S)R′ end group or a —OC(═S)— linking moiety, as these phrases are defined hereinabove, where R′ is as defined herein.

The term “N-carbamate” describes an R″OC(═O)—NR′— end group or a —OC(═O)—NR′— linking moiety, as these phrases are defined hereinabove, with R′ and R″ as defined herein.

The term “O-carbamate” describes an —OC(═O)—NR′R″ end group or an —OC(═O)—NR′— linking moiety, as these phrases are defined hereinabove, with R′ and R″ as defined herein.

The term “O-thiocarbamate” describes a —OC(═S)—NR′R″ end group or a —OC(═S)—NR′— linking moiety, as these phrases are defined hereinabove, with R′ and R″ as defined herein.

The term “N-thiocarbamate” describes an R″OC(═S)NR′— end group or a —OC(═S)NR′— linking moiety, as these phrases are defined hereinabove, with R′ and R″ as defined herein.

The term “S-dithiocarbamate” describes a —SC(═S)—NR′R″ end group or a —SC(═S)NR′— linking moiety, as these phrases are defined hereinabove, with R′ and R″ as defined herein.

The term “N-dithiocarbamate” describes an R″SC(═S)NR′— end group or a —SC(═S)NR′— linking moiety, as these phrases are defined hereinabove, with R′ and R″ as defined herein.

The term “urea”, which is also referred to herein as “ureido”, describes a —NR′C(═O)—NR″R′″ end group or a —NR′C(═O)—NR″— linking moiety, as these phrases are defined hereinabove, where R′ and R″ are as defined herein and R′″ is as defined herein for R′ and R″.

The term “thiourea”, which is also referred to herein as “thioureido”, describes a —NR′—C(═S)—NR″R′″ end group or a —NR′—C(═S)—NR″— linking moiety, with R′, R″ and R′″ as defined herein.

The term “C-amide” describes a —C(═O)—NR′R″ end group or a —C(═O)—NR′— linking moiety, as these phrases are defined hereinabove, where R′ and R″ are as defined herein.

The term “N-amide” describes a R′C(═O)—NR″— end group or a R′C(═O)—N— linking moiety, as these phrases are defined hereinabove, where R′ and R″ are as defined herein.

The term “guanyl” describes a R′R″NC(═N)— end group or a R′NC(═N)— linking moiety, as these phrases are defined hereinabove, where R′ and R″ are as defined herein.

The term “guanidine” describes a R′NC(═N)—NR″R′″ end group or a —R′NC(═N)—NR″— linking moiety, as these phrases are defined hereinabove, where R′, R″ and R′″ are as defined herein.

The term “hydrazine” describes a —NR′—NR″R′″ end group or a —NR′—NR″— linking moiety, as these phrases are defined hereinabove, with R′, R″, and R′″ as defined herein.

The term “silyl” describes a —SiR′R″R′″ end group or a —SiR′R″— linking moiety, as these phrases are defined hereinabove, whereby each of R′, R″ and R′″ are as defined herein.

The term “siloxy” describes a Si(OR′)R″R′″ end group or a —Si(OR′)R″— linking moiety, as these phrases are defined hereinabove, whereby each of R′, R″ and R′″ are as defined herein.

The term “silaza” describes a Si(NR′R″)R′″ end group or a —Si(NR′R″)— linking moiety, as these phrases are defined hereinabove, whereby each of R′, R″ and R′″ is as defined herein.

The term “silicate” describes a —O—Si(OR′)(OR″)(OR′″) end group or a —O—Si(OR′)(OR″)— linking moiety, as these phrases are defined hereinabove, with R′, R″ and R′″ as defined herein.

The term “boryl” describes a —BR′R″ end group or a —BR′— linking moiety, as these phrases are defined hereinabove, with R′ and R″ are as defined herein.

The term “borate” describes a —O—B(OR′)(OR″) end group or a —O—B(OR′)(O—) linking moiety, as these phrases are defined hereinabove, with R′ and R″ are as defined herein.

As discussed herein, the bioavailability enhancing moiety of the hybrid compounds of the present invention is selected or designed such that it increases the aqueous solubility of the compound it forms a part of while maintaining its capacity to cross the BBB. To this end, several polymers which are amphiphilic, i.e., water soluble yet essentially neutral in charge, together with the freedom to select various polymers at various lengths, i.e., number of repeating monomers, or subunits, render these substances as suitable bioavailability enhancing moieties for use in context of the invention.

Hence, according to preferred embodiments of the invention, the bioavailability enhancing moiety is selected from the group consisting of a poly(alkylene glycol), poly(ethylene imine), poly(vinyl alcohol), poly(methyl vinyl ether), poly(n-isopropyl acrylamide), poly(n,n-dimethyl acrylamide), polyacrylamide and poly(2-hydroxyethyl methacrylate). Preferably, the poly(alkylene glycol) is selected from the group consisting of poly(ethylene glycol), poly(propylene glycol) and poly(butylene glycol), and more preferably, the poly(alkylene glycol) is poly(ethylene glycol).

As discussed hereinabove, the bioavailability enhancing moiety can be directly attached to the fullerene moiety or via a first linking moiety. The attachment via a linking moiety may stem from a chemical/synthetic requirement, but may also add two basic advantages to the resulting hybrid compounds; these are: (i) contributing to the bioavailability of the hybrid compound by contributing additional polarizable groups to the compound, and/or (ii) allowing the attachment of more than one bioavailability enhancing moieties to the fullerene moiety by virtue of having more than one functional groups available for attachment with bioavailability enhancing moieties.

As used herein, the phrase “functional group” describes a chemical group that is capable of undergoing a chemical reaction that typically leads to a bond formation. The bond, according to the present invention, is preferably a covalent bond. Chemical reactions that lead to a bond formation include, for example, nucleophilic and electrophilic substitutions, nucleophilic and electrophilic addition reactions, addition-elimination reactions, cycloaddition reactions, rearrangement reactions and any other known organic reactions that involve a reactive group.

Exemplary chemical moieties which can serve as a first linking moiety according to the present invention include, without limitation, a malonic acid residue, a 5,6,7,8-tetrahydronaphthalene-diol residue, a 5,6,7,8-tetrahydro-naphthalene-diol residue, a pyrrolidine residue, an aziridine residue and a phosphonate residue. Preferably the first linking moiety is a malonic acid residue.

As discussed hereinabove, the receptor ligand residue can be directly attached to the bioavailability enhancing moiety or via a second linking moiety. As in the case of the first linking moiety, the attachment via a second linking moiety may stem from a chemical/synthetic requirement, and also add the abovementioned advantages to the resulting hybrid compounds. The second linking moiety may also form as a result of a chemical reaction between a functional group on the glutamate receptor ligand residue and a functional group on the bioavailability enhancing moiety.

Hence, the second linking moiety may be selected from the group consisting of amine, alkyl, alkenyl, cycloalkyl, heteroalicyclic, aryl, heteroaryl, methyleneamine, amine oxide, sulfate, thiosulfate, sulfite, thiosulfite, sulfinate, sulfoxide, sulfonate, S-sulfonamide, N-sulfonamide, disulfide, phosphonate, phosphinyl, phosphine oxide, phosphine sulfide, phosphate, phosphite, thiophosphate, carbonyl, thiocarbonyl, oxime, azo, peroxo, C-carboxylate, O-carboxylate, C-thiocarboxylate, O-thiocarboxylate, N-carbamate, O-carbamate, O-thiocarbamate, N-thiocarbamate, S-dithiocarbamate, N-dithiocarbamate, urea, thiourea, C-amide, N-amide, guanyl, guanidine, hydrazine, hydrazide, thiohydrazide, silyl, siloxy, silaza, silicate, boryl and borate. Preferably, the second linking moiety is C-amide.

As mentioned hereinabove, while reducing the present invention to practice the present inventors have successfully prepared several hybrid compounds as presented hereinabove. Thus, according to further aspects of the present invention, there is provided a method of synthesizing hybrid compounds as presented hereinabove, the method includes two basic steps as follows:

(i) forming an adduct between a bioavailability enhancing moiety and one or more glutamate receptor ligands by reacting a bioavailability enhancing moiety with one or more glutamate receptor ligands to thereby obtain a bioavailability enhancing moiety covalently attached to one or more glutamate receptor ligand residues; and

(ii) forming an adduct between the adduct formed in step (i) and the fullerene moiety by reacting the bioavailability enhancing moiety covalently attached to one or more glutamate receptor ligand residues with a fullerene, thereby obtaining a hybrid compound as presented hereinabove.

This reaction of step (i) may follow any known chemical reaction which is based on forming a covalent bond between two functional groups. As mentioned above, this reaction may include a third compound whereby a residue thereof will act as a second linking moiety between the bioavailability enhancing moiety and the receptor ligand residue. The second linking moiety can also be regarded as the chemical group which is formed as a result of the reaction between the functional group of the bioavailability enhancing moiety and the functional group of the receptor ligand residue.

The reaction of step (ii) between the fullerene moiety and the adduct formed in step (i) may follow known chemical reaction in which fullerenes are substituted and derivatized, as these reactions are known to any artisan skilled in the art. Exemplary reactions according to which fullerenes can be substituted may include, without limitation, a cycloaddition between a fullerene and a bioavailability enhancing moiety having a reactive double bond or a dien moiety such as a 2- and/or -5-substituted-1H-pyrrole residue as a substituent thereof, substituted at position 2 and/or 5; by a radical photoaddition of substituted reactive species such as an N-substituted piperazine; and by reacting previously substituted fullerenes, such as halogenated fullerenes or carboxyfullerenes, which can be regarded as a fullerene moiety attached to a first linking moiety according to the present invention, with the adduct formed in step (i).

Alternatively, one or more bioavailability enhancing moieties can be attached by conventional chemical processes to a first linking moiety, and then be linked to one or more receptor ligands to thereby form an adduct of one or more adducts of step (i), and then attached this structure to the fullerene moiety as described in step (ii) above.

Further alternatively, the bioavailability enhancing moiety can be attached to a receptor ligand residue by conventional chemical processes, and then one or more of these adducts is attached to a first linking moiety to form the abovementioned adduct of adducts, and then be attached to the fullerene moiety by chemical processes similar to that described in step (ii).

In an effort to increase the effect of the bioavailability enhancing moieties and multiply the number of receptor ligand residues present in the hybrid compounds, the fullerene moiety can be attached to more than one bioavailability enhancing moiety-receptor ligand residue adduct, and to more than one adduct of adducts via a first linking moiety, as described herein and demonstrated in the Example section that follows.

These synthetic procedures were successfully demonstrated, as presented in the Example section presented hereinunder as follows:

One hydroxyl end group of polyethyleneglycol was protected by tert-butyldimethylsilyl chloride using imidazole as a base so as to avoid possible polymerization reaction. Thereafter a DCC-mediated coupling reaction of mono-silyl-protected polyethyleneglycol with malonic acid in acetonitrile afforded a malonic acid bis(silyl-protected-polyethyleneglycol) ester. The protecting groups were thereafter removed by tetrabutylammonium fluoride to obtain a free bis-alcohol derivative, followed by a reaction of the bis-alcohol with p-nitrophenylchloroformate in the presence of triethylamine to obtain a bis-p-nitrophenylcarbonate malonic acid (bis-p-nitrophenylcarbonate-polyethyleneglycol) ester. The latter ester compound was coupled with 1-aminoadamantane in DMF, to produce a malonic acid bis(adamantylcarbamate-polyethyleneglycol) ester, which was reacted with a fullerene in the presence of 1,8-diazabicyclo[5.4.0]undec-7-ene (DBU), and I₂ in toluene to produce a 2,2-fullerenyl-malonic acid bis(adamantylcarbamate-polyethyleneglycol) ester, an exemplary hybrid compound of the present invention.

Alternatively, as is further demonstrated in the Examples section hereinunder, a polyethyleneglycol was reacted with adamantylisocyanate in THF to afford an adamantyl-carbamic acid polyethyleneglycol ester. This ester was coupled using DCC with malonic acid in acetonitrile and afforded a malonic acid bis(adamantylcarbamate-polyethyleneglycol) ester which was reacted with a fullerene in the presence of DBU and I₂, to thereby obtain an exemplary hybrid compound of the present invention.

The last DBU-mediated coupling of the fullerene to a malonic acid bis(adamantylcarbamate-polyethyleneglycol) ester can be conducted such that two or more these esters will be attached to the fullerene moiety, as demonstrated in the Examples section that follows wherein two such esters were attached to one fullerene moiety.

As mentioned above, while reducing the present invention to practice, the inventors have designed and successfully prepared various hybrid compounds, as demonstrated and exemplified in the Examples section that follows hereinbelow, using Cₒ fullerenes, diethyleneglycol, tetraethyleneglycol, PEG-400 and PEG-1500 as bioavailability enhancing moieties, malonic acid residues as a first linking moiety and adamantylisocyanate and 1-aminoadamantane as receptor ligand residues, both of the latter formed an amide (not regarding the terminal oxygen of the polyethyleneglycol moiety) as a second linking moiety upon attachment to the polyethyleneglycol moiety.

While further reducing the present invention to practice, additional novel compounds, being intermediates in the syntheses of the hybrid compounds described herein, have been obtained. These compounds, having a bioavailability enhancing moiety linked to one or more glutamate receptor ligand moieties, can also serve as therapeutic, diagnostic and/or research agents.

Hence, according to another aspect of the present invention there are provided compounds having a general Formula V:

MX-Y-Z)_{q} Formula V

wherein:

M is a first linking moiety, as described herein; X is a bioavailability enhancing moiety, as described herein; Y is a second linking moiety, as described herein; Z is a glutamate receptor ligand residue, as described herein; and q is an integer of 1-10, whereby the bioavailability enhancing moiety has a general formula IV as is presented and defined hereinabove.

Exemplary compounds according to preferred embodiments of this aspect of the present invention, which were prepared in the course of preparing the hybrid, fullerene-containing compounds presented hereinabove include, without limitation, Compound 5, Compound 11, Compound 15 and Compound 18. The preparation of these compounds is demonstrated in the Examples section that follows hereinbelow.

A particularly preferred compound in this context of the present invention is the intermediate compound malonic acid bis(adamantylcarbamate-polyethyleneglycol) ester.

As discussed hereinabove, the hybrid compounds of the present in invention have been specifically designed and successfully prepared so as to contain, among other beneficial attributes, three main attributes: being capable of crossing the BBB, capable of acting as antioxidants so to exert a neuroprotective effect, and capable of effecting one or more specific receptors in the CNS, and specifically to act as antagonists for the NMDA receptor and by that exert amelioration of medical conditions which are associated with overactivation thereof, as known to occur in many CNS-related diseases, disorders and trauma. Achieving these capacities are supra to beneficial effects of these compounds in treating other medical conditions in other parts of the body, and on other systems and targets than receptors.

As demonstrated in the Example section that follows, exemplary compounds of the present invention were shown to successfully treat and ameliorate a CNS-associated experimental disease condition of animal models, namely experimental autoimmune encephalomyelitis (EAE) induced in animal models (mice) which simulates the human medical condition of multiple sclerosis, by attenuating the progress of the disease at various stages thereof as measured by qualitative observation of the pathological state of the animal models and qualitative observation of reduced degree of disease-caused axonal damage by various staining methods of spinal cord sections taken from samples of these animal models.

Hence, according to another aspect of the present invention there is provided a method of treating medical conditions in which modulating and/or inhibiting an activity of a glutamate receptor is beneficial, CNS associated diseases, disorders or trauma, oxidative stress associated diseases or disorders, diseases or disorders in which neuroprotection is beneficial, viral infections, bacterial infections, cancer and medical conditions at least partially treatable by the hybrid compounds of the present invention, the method is effected by administering to a subject in need thereof a therapeutically effective amount of a hybrid compound. The hybrid compound utilized in this and other aspects of the present invention comprises a fullerene moiety, one or more bioavailability enhancing moieties and one or more glutamate receptor ligand residues, as presented in detail hereinabove.

Each of the hybrid compounds described herein can therefore be utilized in any of the aspects of the present invention in a form of a pharmaceutically acceptable salt, a prodrug, a solvate and/or a hydrate thereof.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the biological activity and properties of the administered compound.

The term “prodrug” refers to an agent, which is converted into the active compound (the active parent drug) in vivo. Prodrugs are typically useful for facilitating the administration of the parent drug. They may, for instance, be bioavailable by oral administration whereas the parent drug is not. The prodrug may also have improved solubility as compared with the parent drug in pharmaceutical compositions. Prodrugs are also often used to achieve a sustained release of the active compound in vivo. An example, without limitation, of a prodrug would be the hybrid compound, having one or more carboxylic acid moieties, which is administered as an ester (the “prodrug”). Such a prodrug is hydrolysed in vivo, to thereby provide the free compound (the parent drug). The selected ester may affect both the solubility characteristics and the hydrolysis rate of the prodrug, and more importantly, in the context of the present invention, the capacity of the free hybrid compound to cross the BBB.

The term “solvate” refers to a complex of variable stoichiometry (e.g., di-, tri-, tetra-, penta-, hexa-, and so on), which is formed by a solute (the hybrid compound) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

The beneficial characteristics of the hybrid compounds described herein render such compounds highly suitable for use in the treatment of the above-mentioned medical conditions.

The hybrid compounds described herein can thus be beneficially used to treat various oxidative stress associated diseases or disorders and/or related conditions including, without limitation, atherosclerosis, ischemia/reperfusion injuries, restenosis, hypertension, cancer, inflammatory diseases or disorders, acute respiratory distress syndrome (ARDS), asthma, inflammatory bowel disease (IBD), dermal and/or ocular inflammations, arthritis, metabolic diseases or disorders and diabetes.

The hybrid compounds described herein can also be beneficially used to treat various CNS associated diseases, disorders or trauma, and/or related conditions including, without limitation, neurodegenerative diseases or disorders, strokes, brain injuries and/or trauma, multiple sclerosis, amyotrophic lateral sclerosis (ALS), Huntington's Disease, Parkinson's disease, Alzheimer's disease, autoimmune encephalomyelitis, AIDS associated dementia, epilepsy, schizophrenia, pain, anxiety, impairment of memory, decreases in cognitive and/or intellectual functions, deteriorations of mobility and gait, altered sleep patterns, decreased sensory inputs, imbalances in the autonomic nerve system, depression, dementia, confusion, catatonia and delirium.

As used herein, the phrase “therapeutically effective amount” describes an amount of the compound being administered which will relieve to some extent one or more of the symptoms of the disorder being treated, herein the medical conditions as detailed hereinabove. More specifically, a therapeutically effective amount means an amount of the hybrid compounds which is sufficient and effective to prevent, alleviate or ameliorate some or all the symptoms of the medical condition or prolong the survival of the subject being treated.

According to a preferred embodiment of the method according to this aspect of the invention, a therapeutically effective amount of the hybrid compounds described herein can range from about 10 μg per kg of body weight to about 600 μg per kg of body weight per day, and more preferably from about 30 μg per kg of body weight to about 300 μs per Kg of body weight per day, as is demonstrated in the Examples section that follows.

The hybrid compounds described herein can be administered, for example, orally, rectally, intravenously, intraventricularly, topically, intranasally, intraperitoneally, intestinally, parenterally, intraocularly, intradermally, transdermally, subcutaneously, intramuscularly, transmucosally, by inhalation and/or by intrathecal catheter. Preferably, the hybrid compounds are administered orally or intravenously, and optionally rectally, transdermally or by intrathecal catheter, depending on the medical condition and the subject being treated.

By being highly beneficial in treating certain medical conditions, the hybrid compounds described herein can be efficiently used for the preparation of a medicament for treating the abovementioned medical conditions.

In any of the aspects of the present invention, the hybrid compounds described herein, either alone or in combination with any other active agents, can be utilized either per se, or as a part of a pharmaceutical composition.

Hence, according to another aspect of the present invention, there are provided pharmaceutical compositions, which comprise, as an active ingredient, one or more of the hybrid compounds described above and a pharmaceutically acceptable carrier.

The pharmaceutical composition may further comprise an additional active ingredient being capable of treating the medical conditions, as detailed hereinabove.

As used herein a “pharmaceutical composition” or “medicament” refers to a preparation of one or more of the hybrid compounds described herein, with other chemical components such as pharmaceutically acceptable and suitable carriers and excipients. The purpose of a pharmaceutical composition is to facilitate administration of a compound to an organism.

Hereinafter, the term “pharmaceutically acceptable carrier” refers to a carrier or a diluent that does not cause significant irritation to an organism and does not abrogate the biological activity and properties of the administered compound. Examples, without limitations, of carriers are: propylene glycol, cyclodextrins, saline, emulsions and mixtures of organic solvents with water, as well as solid (e.g., powdered) and gaseous carriers.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences” Mack Publishing Co., Easton, Pa., latest edition, which is incorporated herein by reference.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more pharmaceutically acceptable carriers comprising excipients and auxiliaries, which facilitate processing of the hybrid compounds into preparations which, can be used pharmaceutically. Proper formulation is dependent upon the route of administration chosen.

For injection, the hybrid compounds of the invention may be formulated in aqueous solutions, preferably in physiologically compatible buffers such as Hank's solution, Ringer's solution, or physiological saline buffer with or without organic solvents such as propylene glycol, polyethylene glycol.

For transmucosal administration, penetrants are used in the formulation. Such penetrants are generally known in the art.

For oral administration, the hybrid compounds of the invention can be formulated readily by combining the hybrid compounds with pharmaceutically acceptable carriers well known in the art. Such carriers enable the hybrid compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active hybrid compounds doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the hybrid compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration. Preferably, formulations for oral administration further include a protective coating, aimed at protecting or slowing enzymatic degradation of the preparation in the GI tract.

For buccal administration, the compositions may take the form of tablets or lozenges formulated in conventional manner.

For administration by inhalation, the hybrid compounds for use according to the present invention are conveniently delivered in the form of an aerosol spray presentation (which typically includes powdered, liquified and/or gaseous carriers) from a pressurized pack or a nebulizer, with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the hybrid compounds and a suitable powder base such as, but not limited to, lactose or starch.

The hybrid compounds described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the hybrid compounds preparation in water-soluble form. Additionally, suspensions of the hybrid compounds may be prepared as appropriate oily injection suspensions and emulsions (e.g., water-in-oil, oil-in-water or water-in-oil in oil emulsions). Suitable lipophilic solvents or vehicles include fatty oils such as sesame oil, or synthetic fatty acids esters such as ethyl oleate, triglycerides, liposomes or Cremophor® and various cremophor-like compounds (nonionic solubilizers and emulsifiers produced by reacting castor oil or other oils with ethylene oxide in various molar ratios). Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the hybrid compounds to allow for the preparation of highly concentrated solutions.

Alternatively, the hybrid compounds may be in powder form for constitution with a suitable vehicle, e.g., sterile, pyrogen-free water, before use.

The hybrid compounds, described herein, may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose, described hereinabove as a therapeutically effective amount.

Determination of a therapeutically effective amount is well within the capability of those skilled in the art, especially in light of the detailed disclosure provided herein.

For any hybrid compounds used in the methods of the invention, the therapeutically effective amount or dose can be estimated initially from activity assays in animals. For example, a dose can be formulated in animal models, as demonstrated in the Examples section that follows, to achieve a circulating concentration range that includes the IC₅₀ as determined by activity assays (e.g., the concentration of the test hybrid compounds, which achieves a half-maximal reduction of the mean arterial blood pressure). Such information is presented hereinbelow in the Examples section that follows, can be used to more accurately determine useful doses in humans.

As is demonstrated in the Examples section that follows, a therapeutically effective amount for the hybrid compounds may range between about 10 μg per Kg of body weight to about 600 μg per Kg of body weight per day.

Toxicity and therapeutic efficacy of the hybrid compounds described herein can be determined by standard pharmaceutical procedures in experimental animals, e.g., by determining the EC₅₀, the IC₅₀ and the LD₅₀ (lethal dose causing death in 50% of the tested animals) for a subject hybrid compound. The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See, e.g., Fingl et al., 1975, in “The Pharmacological Basis of Therapeutics”, Ch. 1 p. 1).

Dosage amount and interval may be adjusted individually to provide plasma levels of the active moiety which are sufficient to maintain the desired effects, termed the minimal effective concentration (MEC). The MEC will vary for each preparation, but can be estimated from in vitro data; e.g., the concentration necessary to achieve 50-90% vasorelaxation of contracted arteries. Dosages necessary to achieve the MEC will depend on individual characteristics and route of administration. HPLC and LC-MS assays or bioassays can be used to determine plasma concentrations.

Dosage intervals can also be determined using the MEC value. Preparations should be administered using a regimen, which maintains plasma levels above the MEC for 10-90% of the time, preferable between 30-90% and most preferably 50%-90%.

Depending on the severity and responsiveness of the condition to be treated, dosing can also be a single administration of a slow release composition described hereinabove, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA (the U.S. Food and Drug Administration) approved kit, which may contain one or more unit dosage forms containing the active ingredient. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a hybrid compound of the present invention formulated in a compatible pharmaceutical carrier may also be prepared, placed in an appropriate container, and labeled for treatment of an indicated condition or diagnosis, as is detailed hereinabove.

Thus, according to a preferred embodiment of the present invention, the pharmaceutical composition described herein is packaged in a packaging material and to identified in print, in or on the packaging material, for use in the treatment of a medical condition selected from the group consisting of a medical condition in which modulating and/or inhibiting an activity of a glutamate receptor is beneficial, a CNS associated disease or, disorder or trauma, an oxidative stress associated disease or disorder, a disease or disorder in which neuroprotection is beneficial, a viral infection, a bacterial infection, cancer and a medical condition at least partially treatable by the hybrid compound.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

The experimental examples presented below describe the preparation of exemplary hybrid compounds, as presented hereinabove, in the form of fullerene-polyethyleneglycol-adamantyl hybrid compounds, according to preferred embodiments. Further presented is a qualitative and quantitive evaluation of the influence of the polyethyleneglycol chain length which serves as a bioavailability enhancing moiety, and the number of adamantly-polyethyleneglycol adduct moieties attached to a fullerene residue on the aqueous solubility of these exemplary hybrid compounds.

### Chemical Syntheses

The following describes two general synthetic routes for the preparation of different fullerene-polyethyleneglycol-adamantyl hybrid compounds according to the present invention, in which adamantyl groups were connected to a fullerene residue via a malonic acid linking moiety through various lengths of polyethyleneglycol moieties. The two procedures converge at the formation of a malonic acid bis-adamantyl-polyethyleneglycol ester adduct before the attachment thereof to the fullerene residue.

Materials and Instrumental Data

All solvents were of analytical grade or better. Toluene and THF were distilled over sodium/benzophenone; other solvents were purchased as anhydrous

Fullerenes were purchased from SES Research, Houston, Tex., USA.

All operations with oxygen-reactive and/or moisture-sensitive compounds were performed according to the Schlenk techniques under argon atmosphere and stored under same.

¹H and ¹³C NMR spectra were recorded on 400 MHz spectrometers in CDCl₃. ¹H and ¹³C NMR signals are reported in ppm. ¹H signals are referenced to the residual proton (7.26 ppm for CDCl₃) of a deuterated solvent and ¹³C NMR signals are referenced to CDCl₃ (77.36 ppm). ¹³C NMR spectra interpretations were supported by DEPT experiments.

Mass spectra were obtained on a spectrometer equipped with CI, EI and FAB probes and on spectrometer equipped with an electrospray ionization mass spectrometry probe (ESI-MS). HRMS results were obtained on MALDI-TOF and ESI mass spectrometers.

IR spectra were recorded on FTIR spectrometer.

Progress of reactions was monitored by TLC on silica gel, visualized by UV-light or developed in iodine chamber.

Flash chromatography was carried out on silica gel (0.04-0.063 mm).

Methods

For clarity of the schemes presented below, a C₆₀ fullerene is depicted as follows:

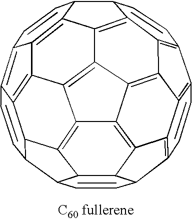

This schematic representation does not attempt to provide a three-dimensional representation of the fullerene moiety nor does it attempt to provide bonding information at the individual atom level. Accordingly, a carboxyfullerene, a tri-malonic acid derivative of C₆₀ is depicted as follows:

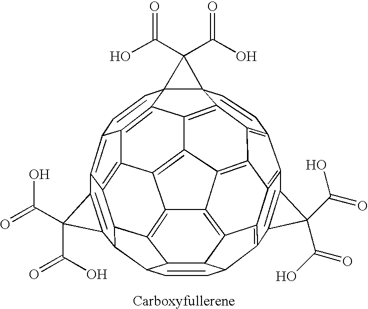

Preparation of Fullerene-Polyethyleneglycol-Adamantyl Hybrid Compounds—General Procedure I:

General Procedure I was based on initial construction of malonate polyethyleneglycol esters terminated with adamantylcarbamates that were further coupled to C₆₀ fullerene, following the Bingel-Hirsch methodology [Lamparth, I. and Hirsch, A., J. Chem. Soc., Chem. Commun. 1994, 1727] as depicted in Scheme 1 below.

A polyethyleneglycol was reacted with tert-butyldimethylsilyl chloride (TBS-Cl) or tert-butyldiphenylsilyl chloride at 0° C. in DMF, using imidazole as a base so as to avoid possible polymerization reaction by protecting one of the terminal hydroxyl groups of the polyethyleneglycol.

DCC-mediated coupling of mono-silyl-protected poly ethyleneglycol (Compound I) with malonic acid in acetonitrile, afforded a malonic acid bis(silyl-protected-polyethyleneglycol) ester (Compound II).

Compound II converted to the corresponding diol (Compound III), by deprotection with tetrabutylammonium fluoride (TBAF) at 0° C. in THF.

Bis-p-nitrophenylcarbonate (Compound IV) was obtained by reacting Compound III with p-nitrophenylchloroformate at 0° C. in THF, using triethylamine as a base.

Compound IV was coupled with 1-aminoadamantane in DMF, to produce malonic acid bis(adamantylcarbamate-polyethyleneglycol) ester (Compound V).

Compound V was reacted with 1,8-diazabicyclo[5.4.0]undec-7-ene (DBU), fullerene (C₆₀) and I₂ in toluene for 36-72 hours under an argon atmosphere and the reaction mixture was purified by flash chromatography silica column to afford the desired 2,2-fullerenyl-malonic acid bis(adamantylcarbamate-polyethyleneglycol) ester (Compound VI).

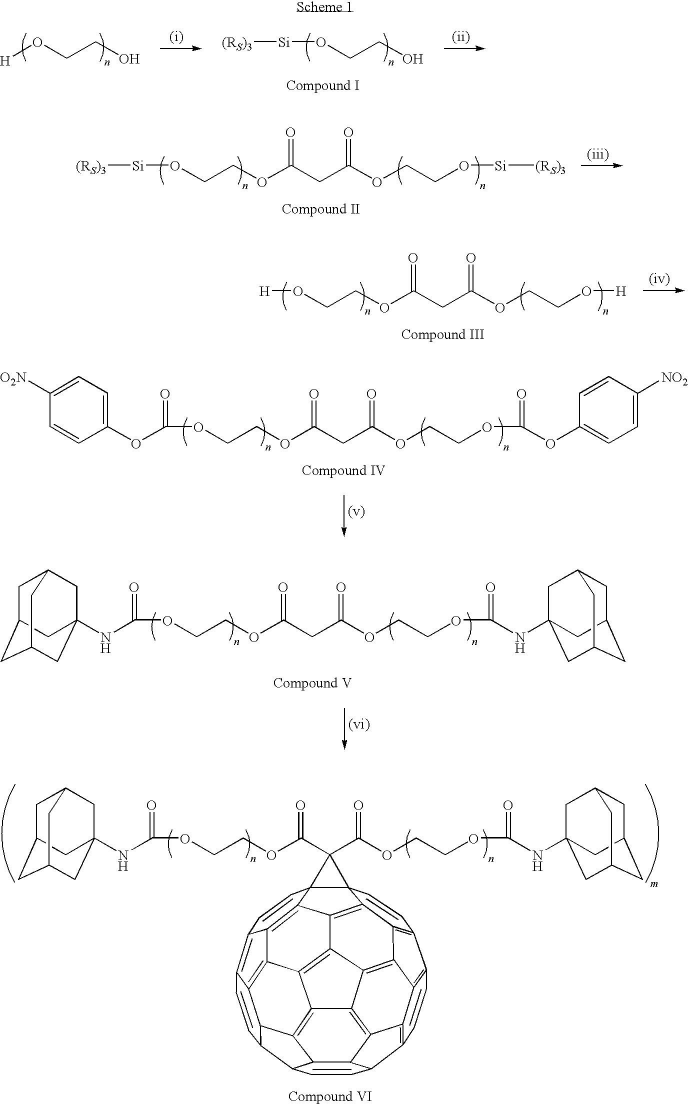

whereas:
(i)=trialkylsilyl/triarylsilyl chloride, imidazole, DMF, 0° C.;
(ii)=malonic acid, DCC, acetonitrile;
(iii)=tetrabutylammonium fluoride, THF, 0° C.;
(iv)=p-nitrophenylchloroformate, triethylamine, THF, 0° C.;
(v)=aminoadamantane, triethylamine, DMF;
(vi)=C₆₀, I₂, DBU, toluene, room temperature;
Rs is alkyl or aryl;
n=1-50;
and m=1-6.

The preparation of an exemplary fullerene-polyethyleneglycol-adamantane hybrid compound of the present invention according to General Procedure I, wherein n=3, is presented below.

### Preparation of Tert-Butyl-Dimethyl-Silanyloxy Tetraethyleneglycol (Compound 1)

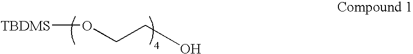

A solution of imidazole (7.0 grams, 102.8 mmol) and tetraethyleneglycol (30.0 grams, 154.4 mmol) in dry DMF (70 ml) was cooled to 0° C. and stirred for 30 minutes under argon atmosphere. Tertbutyldimethylsilyl chloride (15.5 g, 102.8 mmol) in dry DMF (50 ml) was added dropwise to the solution, and stirring continued for two additional hours at 0° C. Thereafter the reaction mixture was allowed to warm to room temperature, water (900 ml) was added and the resulting solution was extracted with ethyl acetate (4 portions of 400 ml). The combined organic extracts were washed with brine and the solvent was evaporation under reduced pressure to give a crude product. The crude product was purified by flash chromatography on silica using ethyl acetate as eluent to give Compound 1 (19.2 grams, 61% yield) as a light yellow oil.

¹H NMR (400 MHz, CDCl₃): δ=3.66 (m, 16H, CH₂—O), 0.86 (s, 9H, CH₃—C), 0.03 (s, 6H, CH₃—Si);

¹³C NMR (100 MHz, CDCl₃): δ=73.0, 72.8, 71.0, 70.9, 63.0, 62.0, 26.2, 18.6, −5.0;

IR (neat): 778, 836, 943, 1107, 1253, 1355, 1467, 1648, 2860, 2930, 3445 cm⁻¹;

MS (CI⁺): m/z 308 (MH⁺).

### Preparation of malonic acid bis(tert-butyl-dimethyl-silanyloxy-tetraethyleneglycol) ester (Compound 2)

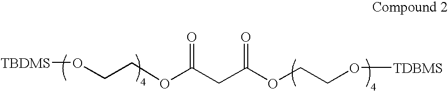

Compound 1 (2.0 grams, 6.5 mmol) dissolved in dry acetonitrile (9 ml) was added to a solution of malonic acid (0.31 grams, 2.9 mmol) followed by the dropwise addition of a solution of DCC (1.4 grams, 6.5 mmol) in dry acetonitrile (7 ml) over a time period of 20 minutes under argon atmosphere. The reaction mixture was stirred for additional 20 minutes during which a white precipitate was formed. The precipitate was filtered, washed with three portions of acetonitrile (20 ml) and the combined organic phase was evaporated under reduced pressure.

The crude product was purified by flash chromatography on a silica column using ethyl acetate:hexanes (65%:35%) as eluent to give Compound 2 (1.61 grams, 81% yield) as a light yellow oil.

Compound 2 is also termed malonic acid bis-[2-(2-{2-[2-(tert-butyl-dimethyl-silanyloxy)-ethoxy]-ethoxy}-ethoxy)-ethyl]ester.

¹H NMR (400 MHz, CDCl₃): δ=4.29 (t, J=4.8 Hz, 4H, CH₂—O—CO), 3.76 (t, J=5.6 Hz, 4H, CH₂—O), 3.70 (t, J=4.8 Hz, 4H, CH₂—O), 3.64 (m, 16H, CH₂—O), 3.55 (t, J=5.6 Hz, 4H, CH₂—O), 3.44 (s, 2H, CH₂—CO), 0.89 (s, 9H, CH₃—C), 0.06 (s, 6H, CH₃—Si);

¹³C NMR (100 MHz, CDCl₃): δ=166.7, 72.9, 71.0, 70.9, 69.1, 64.8, 63.0, 41.5, 26.2, 18.6, −5.0;

IR (neat): 774, 837, 947, 1108, 1253, 1466, 1744, 2860, 2933 cm⁻¹;

MS (CI): m/z 685.3 (MH⁺).

### Preparation of malonic acid bis(tetraethyleneglycol)ester (Compound 3)

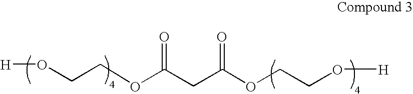

Compound 2 (6.53 grams, 9.5 mmol) was dissolved in THF (50 ml) and added by syringe to a solution of tetrabutylammonium fluoride in THF (24 ml of 1M solution) at 0° C. After stirring for 2 hours at 0° C., the reaction mixture was allowed to warm to room temperature and stirred for additional 30 minutes. Thereafter methylene chloride (400 ml) was added and the mixture was washed with three portions of saturated Na₂SO₄ aqueous solution (50 ml), the aqueous solution was extracted with three portions of methylene chloride (50 ml), and combined organic phase was evaporated under reduced pressure to afford a crude product.

The crude product was purified by flash chromatography on silica using methylene chloride:methanol (9:1) as eluent to give Compound 3 (0.27 grams, 91% yield) as a light yellow oil.

Compound 3 is also termed malonic acid bis-(2-{2-[2-(2-hydroxy-ethoxy)-ethoxy]-ethoxy}-ethyl)ester.

¹H NMR (400 MHz, CDCl₃): δ=4.20 (t, J=4.8 Hz, 4H, CH₂—O—CO), 3.61 (m, 4H, CH₂—O), 3.61 (m, 4H, CH₂—O), 3.56 (m, 16H, CH₂—O), 3.49 (t, J=5.2 Hz, 4H, CH₂—O), 3.36 (s, 2H, CH₂—CO);

¹³C NMR (100 MHz, CDCl₃): δ=167.6, 72.7, 70.7, 70.6, 70.5, 64.6, 61.6, 41.3:

IR (neat): 940, 1104, 1283, 1340, 1458, 1636, 1743, 2876, 3387 cm⁻¹;

MS (CI): m/z 457.1 (MH⁺).

### Preparation of malonic acid bis(4-nitro-phenoxycarboxylate-tetraethyleneglycol)ester (Compound 4)

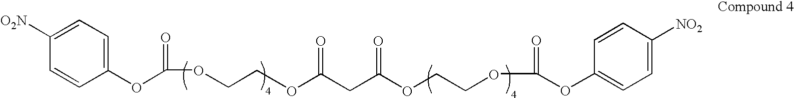

A solution of Compound 3 (1.10 grams, 2.41 mmol) and triethylamine (1.9 ml) in dry THF (100 ml) was cooled to 0° C. under argon atmosphere and a solution p-nitrophenylchloroformate (1.07 grams, 5.30 mmol) in dry THF (40 ml) was added dropwise thereto during one hour. Thereafter, the reaction mixture was allowed to warm up to room temperature, and stirred for 2 hours while monitoring the reaction progress by TLC using ethyl acetate as eluent. The formed precipitate was collected by filtration and dried under reduced pressure to afford a crude product.

The crude product was purified by flash chromatography on silica using ethyl acetate as eluent to give Compound 4 (1.33 grams, 70% yield) as a yellow oil. Compound 4 is also termed malonic acid bis-[2-(2-{2-[2-(4-nitro-phenoxycarbonyloxy)-ethoxy]-ethoxy}-ethoxy)-ethyl]ester).

¹H NMR (400 MHz, CDCl₃): δ=8.22 (d, J=9.2 Hz, 2H, H—Ar), 7.35 (d, J=9.2 Hz, 4H, H—Ar), 4.39 (t, J=4.4 Hz, 4H, CH₂—O—CO—O), 4.25 (t, J=4.8 Hz, 4H, CH₂—O—CO), 3.77 (t, J=4.8 Hz, 4H, CH₂—O—CO), 3.67 (m, 12H, CH₂—O), 3.61 (m, 8H, CH₂—O), 3.40 (s, 2H, CH₂—CO);

¹³C NMR (100 MHz, CDCl₃): δ=166.7, 155.7, 152.7, 145.6, 125.5, 122.0, 70.9, 70.81, 70.76, 69.0, 68.8, 68.5, 64.7, 41.4;

IR (neat): 664, 774, 860, 1214, 1349, 1491, 1524, 1592, 1615, 1753 cm¹;

MS (CI): m/z 787.0 (MH⁺).

### Preparation of malonic acid bis(adamantan-1-ylcarbamate-tetraethyleneglycol) ester (Compound 5)

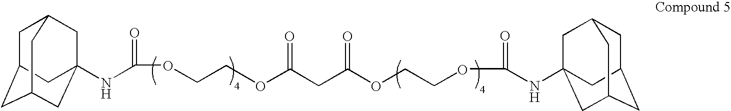

Triethylamine (2 ml) and Compound 4 (1.5 grams, 1.9 mmol) were added to a solution of 1-adamantylamine (0.634 grams, 4.19 mmol) in dry DMF (8 ml) at room temperature. The reaction progress was monitored by TLC using methylene chloride:methanol (95%:5%) as eluent; following a species having R_{f} of 0.6. After the reaction was completed, the DMF was removed under reduced pressure to afford a crude product.

The crude product was purified by flash chromatography on silica using methylene chloride:methanol, (95%:5%) as eluent to give Compound 5 (1.14 grams, 74%) as a light yellow oil.

Compound 5 is also termed malonic acid bis-[2-(2-{2-[2-(adamantan-1-ylcarbamoyloxy)-ethoxy]ethoxy}-ethoxy)-ethyl]ester.

¹H NMR (400 MHz, CDCl₃): δ=4.70 (broad s, 2H, NH), 4.23 (t, J=4.8 Hz, 4H, CH₂—O—CO), 4.08 (t, J=4.0 Hz, 4H, CH₂—O—CO—NH), 3.65 (t, J=5.2 Hz, 4H, CH₂—O) 3.59 (m, 20H, CH₂—O), 3.39 (s, CH₂), 2.00 (m, 6H, CH), 1.86 (d, J=2.8 Hz, 12H, CH₂), 1.60 (t, J=2.8 Hz, 12H, CH₂);

¹³C NMR (100 MHz, CDCl₃): δ 166.6, 154.4, 70.7, 70.6, 69.9, 69.0, 64.7, 63.2, 50.8, 41.9, 41.4, 36.5, 29.6;

IR (CHCl₃): 1067, 1139, 1277, 1295, 1456, 1508, 1723, 2853, 2912 cm⁻¹;

MS (FAB⁺): m/z 833.5 (MNa⁺), 849.0 (MK⁺).

### Preparation of 2,2-fullerenyl-malonic acid bis(adamantan-1-ylcarbamate-tetraethyleneglycol) ester (Compound 6)

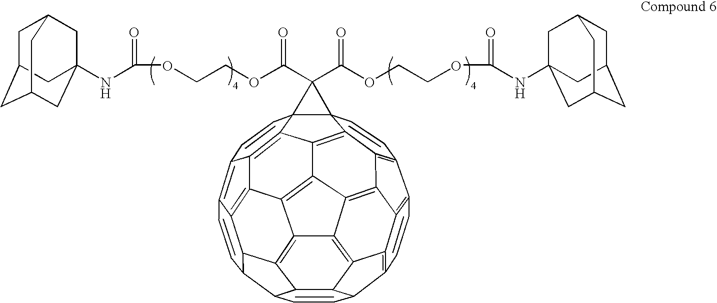

DBU (0.47 grams, 3.08 mmol) was dissolved in toluene (30 ml) and added to a stirred solution of Compound 5 (1.0 gram, 1.23 mmol), C₆₀ (0.9 grams, 1.23 mmol) and I₂ (0.3 grams, 1.23 mmol) in toluene (310 ml), and the mixture was stirred for 36 hours under argon atmosphere at room temperature. Thereafter the reaction mixture was loaded on top of short flash chromatography column packed with silica and eluted with toluene to remove excess fullerene. Further elution with toluene:isopropanol (99:1) gave Compound 6 (0.83 grams, 44% yield) as dark brown solid.

¹H NMR (400 MHz, CDCl₃): δ=4.63 (broad s, 2H, NH), 4.63 (t, J=4.8 Hz, 4H, CH₂—O—CO), 4.11 (t, J=4.0 Hz, 4H, CH₂—O—CO—NH), 3.85 (t, J=4.8 Hz, 4H, CH₂—O) 3.62 (m, 20H, CH₂—O), 2.03 (m, 6H, CH), 1.88 (d, J=2.4 Hz, 12H, CH₂), 1.62 (m, 12H, CH₂);

¹³C NMR (100 MHz, CDCl₃): δ=163.8, 154.5, 145.6, 145.5, 145.2, 145.0, 144.9, 144.2, 143.4, 143.3, 143.2, 142.5, 142.2, 141.2, 139.4, 71.8, 71.01, 71.00, 70.9, 70.8, 69.1, 66.6, 63.4, 51.0, 42.1, 36.6, 29.7;

IR (KBr): 524, 704, 804, 1025, 1098, 1263, 1449, 1714, 2907, 2963 cm⁻¹;

MS (MALDI-TOF): m/z 1552.4 (MNa⁺);

λₘₐₓ (CHCl₃): 257, 326, 424, 475, 683 nm.

### Preparation of Tert-Butyl-Diphenyl-Silanyloxy Polyethyleneglycol Wherein the Polyethyleneglycol is PEG-1500 (Compound 7)

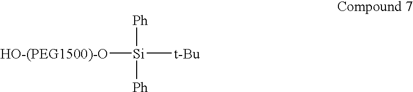

Tert-butyldiphenylsilyl chloride (1.4 grams, 5.0 mmol) in DMF (5 ml) was added dropwise to a stirred solution of and a polyethyleneglycol, commonly known as PEG-1500, having an average of 34 ethyleneglycol units in each polyethyleneglycol chain and an average molecular weight of about 1500 grams per mole (12.0 grams, 8.0 mmol) and imidazole (0.54 grams, 8.0 mmol) in dry DMF (40 ml) under argon atmosphere at room temperature. The solution was stirred at room temperature for 18 hours, and thereafter the DMF was removed under reduced pressure and the product was purified by two flash chromatography columns on silica using methylene chloride:methanol (9:1) to give Compound 7 (3.72 grams, 43% yield) as a white solid oil.

¹H NMR (400 MHz, CDCl₃): δ=7.64 (m, 4H, CH), 7.35 (m, 6H, CH), 3.62 (m, ˜136H, CH₂—O), 1.02 (s, 9H, CH₃—C);

¹³C NMR (100 MHz, CDCl₃): δ=135.8, 129.8, 127.9, 70.8, 63.9, 62.0, 27.1, 19.4;

MS (ESI⁺): m/z 1745.7 (Average Mw).

### Preparation of malonic acid bis(tert-butyl-dimethyl-silanyloxy-PEG-1500) ester (Compound 8)

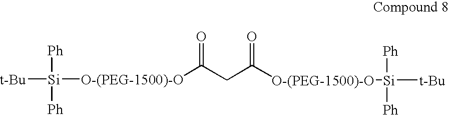

Compound 7 (3.5 grams, 2.0 mmol) dissolved in dry acetonitrile (15 ml) was added to a solution of malonic acid (0.1 grams, 1 mmol) followed by the dropwise addition of a solution of DCC (0.42 grams, 2.0 mmol) in dry acetonitrile (8 ml) over a time period of 20 minutes under argon atmosphere. The reaction mixture was stirred for 40 hours during which a white precipitate was formed. The precipitate was filtered, washed with three portions of methylene chloride (20 ml) and the combined organic phase was evaporated under reduced pressure.

The crude product was purified by flash chromatography on a silica column using methylene chloride:methanol (9:1) as eluent to give Compound 8 (0.57 grams, 16% yield) as an off white wax.

¹H NMR (400 MHz, CDCl₃): δ=7.65 (m, 8H, CH), 7.36 (m, 12H, CH), 4.27 (t, J=5.6 Hz, 4H, CH₂—OCO), 3.78 (t, J=5.2 Hz, 4H, CH₂—O) 3.62 (m, ˜272H, CH₂—O), 3.40 (s, 2H, CH₂) 1.02 (s, 9H, CH₃);

¹³C NMR (100 MHz, CDCl₃): δ=136.6, 134.7, 130.6, 128.7, 71.6, 65.6, 64.5, 42.3, 27.9, 20.2

MS (ESI⁺): m/z 3518.7 (Average Mw).

### Preparation of Malonic Acid Bis(PEG-1500) Ester (Compound 9)

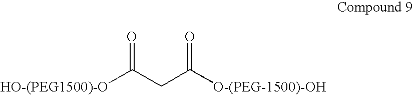

Compound 8 is dissolved in THF and added by syringe to a solution of tetrabutylammonium fluoride in THF at 0° C. After stirring for 4 hours at 0° C., the reaction mixture is allowed to warm to room temperature and stirred for additional hour. Thereafter methylene chloride is added and the mixture is washed with three portions of saturated Na₂SO₄ aqueous solution, the aqueous solution is extracted with three portions of methylene chloride, and combined organic phase is evaporated under reduced pressure to afford the crude product.

The crude product is purified by flash chromatography on silica using methylene chloride:methanol (9:1) as eluent to give Compound 9.

### Preparation of malonic acid bis(4-nitro-phenoxycarboxylate-PEG-1500) ester (Compound 10)

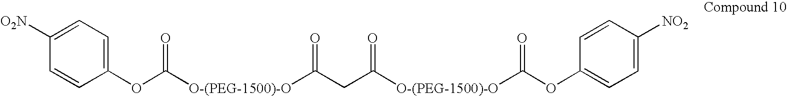

A solution of Compound 9 and triethylamine in dry THF is cooled to 0° C. under argon atmosphere and a solution p-nitrophenylchloroformate in dry THF is added dropwise thereto during one hour. Thereafter, the reaction mixture is allowed to warm up to room temperature, and stirred for 2 hours while monitoring the reaction progress by TLC using ethyl acetate as eluent. The formed precipitate is collected by filtration and dried under reduced pressure to afford a crude product.

The crude product is purified by flash chromatography on silica using ethyl acetate as eluent to give Compound 4.

### Preparation of malonic acid bis(adamantan-1-ylcarbamate-PEG-1500) ester (Compound 11)

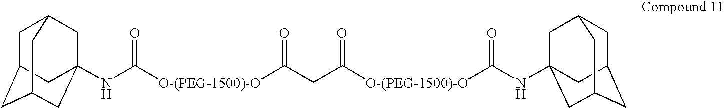

Triethylamine and Compound 10 are added to a solution of 1-adamantylamine in dry DMF at room temperature. The reaction progress is monitored by TLC using methylene chloride:methanol (95%:5%) as eluent. After the reaction is completed, the DMF is removed under reduced pressure to afford a crude product.

The crude product is purified by flash chromatography on silica using methylene chloride:methanol, (95%:5%) as eluent to give Compound 11.

### Preparation of 2,2-fullerenyl-malonic acid bis(adamantan-1-ylcarbamate-PEG-1500) ester (Compound 12)

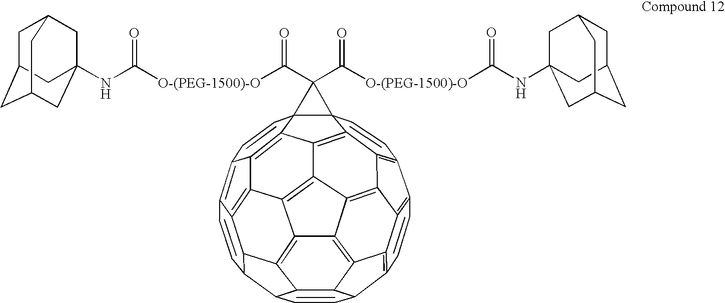

DBU is dissolved in toluene and added to a stirred solution of Compound 11, C₆₀ and I₂ in toluene, and the mixture is stirred for 72 hours under argon atmosphere at room temperature. Thereafter the reaction mixture is loaded on top of short flash chromatography column packed with silica and eluted with toluene to remove excess fullerene. Further elution with toluene:isopropanol (99:1) gives Compound 12.

Preparation of Fullerene-Polyethyleneglycol-Adamantyl Hybrid Compounds—General Procedure II:

General Procedure II was used for preparation of target hybrid compounds of the present invention without use of protection groups, and was based on reaction of polyethyleneglycols, such as PEG-400, with adamantylisocyanate to produce a series of adamantyl-carbamic acid polyethyleneglycol esters, which were further coupled, as described in General Procedure I above, to malonic acid and C₆₀, as depicted in Scheme 2 below.

A polyethyleneglycol was reacted with adamantylisocyanate under reflux conditions in THF to afford an adamantyl-carbamic acid polyethyleneglycol ester (Compound VII).

DCC-mediated coupling of Compound VII with malonic acid in acetonitrile, afforded a malonic acid bis(adamantylcarbamate-polyethyleneglycol) ester (Compound V).

Compound V was reacted with DBU, C₆₀ and I₂ in toluene essentially as described in General Procedure I hereinabove to afford the desired 2,2-fullerenyl-malonic acid bis(adamantylcarbamate-polyethyleneglycol) ester (Compound VI).

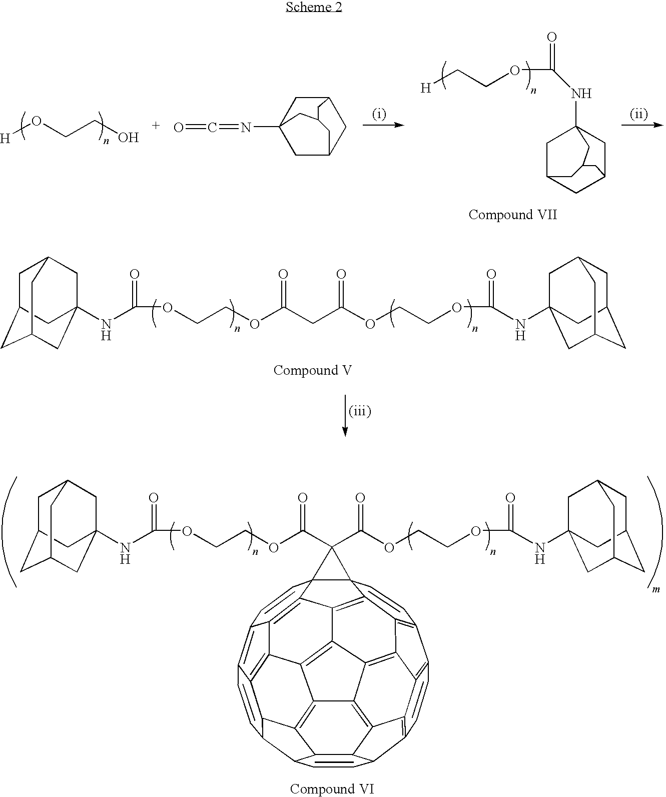

whereas:
(i)=THF, reflux;
(ii)=malonic acid, DCC, acetonitrile;
(iii)=C₆₀, I₂, DBU, toluene, room temperature;
n=1-50;
and m=1-6.

Following are presented synthetic procedures of exemplary fullerene-polyethyleneglycol-adamantane hybrid compounds of the present invention as prepared according to General Procedure II.

### Preparation of adamantan-1-yl-carbamic acid tetraethyleneglycol ester (Compound 13)

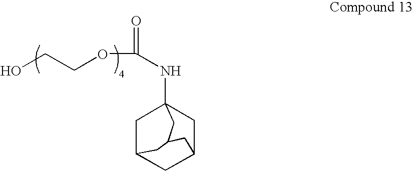

A mixture of 1-adamantylisocyanate (2.0 grams, 11.3 mmol) and tetraethyleneglycol (4.4 grams, 22.6 mmol) in dry THF (30 ml) was refluxed for 20 hours under argon atmosphere. After cooling down to room temperature, the solvent was evaporated under reduced pressure and the crude product was purified by flash chromatography on silica using methylene chloride:methanol (92%:8%) as eluent to give Compound 13 (3.46 grams, 82% yield) as a colorless oil.

¹H NMR (400 MHz, CDCl₃): δ=4.14 (t, J=8.8 Hz, 2H, CH₂—O—CO), 3.72 (t, J=4.0 Hz, 2H, CH₂—O), 3.67 (m, 10H, CH₂—O), 3.61 (t, J=4.0 Hz, 2H, CH₂—O), 2.06 (m, 3H, CH), 1.91 (d, J=2.8 Hz, 6H, CH₂), 1.65 (t, J=2.8 Hz, 6H, CH₂);

¹³C NMR (100 MHz, CDCl₃): δ=154.5, 72.8, 70.7, 70.6, 70.5, 70.4, 69.9, 63.2, 61.8, 50.8, 41.9, 36.5, 29.6

IR (neat): 943, 1067, 1232, 1360, 1455, 1535, 1708, 2852, 2902, 3437 cm⁻¹;

MS (FAB⁺): m/z 372.2 (MH⁺), 394.2 (MNa⁺), 410.0 (MK⁺).

### Preparation of Compound 5 from Compound 13

A solution of DCC (1.10 grams, 5.4 mmol) in dry acetonitrile (7 ml) was added dropwise to a solution of malonic acid (0.25 grams, 2.45 mmol) and Compound 13 (2.0 grams, 5.4 mmol) in dry acetonitrile (20 ml) over a time period of 20 minutes under argon atmosphere. The reaction mixture was stirred for additional 20 minutes during which a white precipitate was formed. The precipitate was filtered, washed with three portions of methylene chloride (20 ml) and combined organic phase was evaporated under reduced pressure to afford a crude product.

The crude product was purified by flash chromatography on silica using methylene chloride:methanol, (95%:5%) as eluent to give Compound 5 (1.32 grams, 65% yield) as a light yellow oil.

¹H NMR (400 MHz, CDCl₃): δ=4.70 (broad s, 2H, NH), 4.23 (t, J=4.8 Hz, 4H, CH₂—O—CO), 4.08 (t, J=4.0 Hz, 4H, CH₂—O—CO—NH), 3.65 (t, J=5.2 Hz, 4H, CH₂—O) 3.59 (m, 20H, CH₂—O), 3.39 (s, CH₂), 2.00 (m, 6H, CH), 1.86 (d, J=2.8 Hz, 12H, CH₂), 1.60 (t, J=2.8 Hz, 12H, CH₂);

¹³C NMR (100 MHz, CDCl₃): δ=166.6, 154.4, 70.7, 70.6, 69.9, 69.0, 64.7, 63.2, 50.8, 41.9, 41.4, 36.5, 29.6;

IR (CHCl₃): 1067, 1139, 1277, 1295, 1456, 1508, 1723, 2853, 2912 cm⁻¹;

MS (FAB⁺): m/z 833.5 (MNa⁺), 849.0 (MK⁺).

### Preparation of adamantan-1-yl-carbamic acid diethyleneglycol ester (Compound 14)

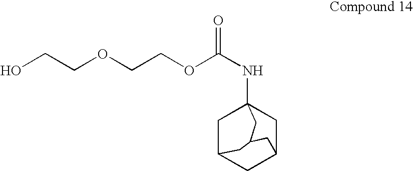

A mixture of 1-adamantylisocyanate (2.0 grams, 11.3 mmol) and diethyleneglycol (2.4 grams, 22.6 mmol) in dry THF (30 ml) was refluxed for 20 hours under argon atmosphere. After cooling down to room temperature, the solvent was evaporated under reduced pressure and the crude product was purified by flash chromatography on silica using methylene chloride:methanol (95%:5%) as eluent to give Compound 14 (2.8 grams, 87% yield) as a colorless oil.

¹H NMR (400 MHz, CDCl₃): δ=4.73 (broad s, 1H, NH), 4.14 (t, J=4.4 Hz, 2H, CH₂—O—CO), 3.70 (t, J=4.4 Hz, 2H, CH₂—O), 3.64 (t, J=4.8 Hz, 2H, CH₂—O), 3.57 (t, J=4.8 Hz, 2H, CH₂—O), 2.04 (m, 3H, CH), 1.89 (d, J=2.8 Hz, 6H, CH₂), 1.63 (t, J=2.8 Hz, 6H, CH₂);

¹³C NMR (100 MHz, CDCl₃): δ=154.7, 72.6, 70.0, 63.2, 61.9, 51.0, 42.0, 36.5, 29.7. IR (CHCl₃): 1279, 1295, 1361, 1456, 1508, 1719, 2853, 2913 cm⁻¹;

MS (FAB⁺): m/z 284.2 (MH⁺), 306.2 (MNa⁺).

### Preparation of malonic acid bis(adamantan-1-ylcarbamate-diethyleneglycol) ester (Compound 15)

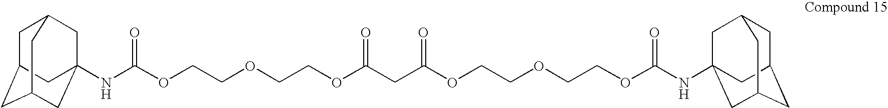

A solution of DCC (1.45 grams, 7.06 mmol) in dry acetonitrile (7 ml) was added dropwise to a solution of malonic acid (0.33 grams, 3.21 mmol) and Compound 14 (2.0 grams, 7.06 mmol) in dry acetonitrile (10 ml) over a time period of 20 minutes under argon atmosphere. The reaction mixture was stirred for additional 20 minutes during which a white precipitate was formed. The precipitate was filtered, washed with three portions of methylene chloride (20 ml) and combined organic phase was evaporated under reduced pressure to afford a crude product.

The crude product was purified by flash chromatography on silica using ethyl acetate:hexanes (3:2) as eluent to give Compound 15 (1.32 grams, 65% yield) as a light yellow oil.

NMR (400 MHz, CDCl₃): δ=4.74 (broad s, 2H, NH), 4.30 (t, J=4.8 Hz, 4H, CH₂—O—CO), 4.14 (t, J=4.4 Hz, 4H, CH₂—O—CO—NH), 4.71 (t, J=4.8 Hz, 4H, CH₂—O), 3.66 (t, J=4.8 Hz, 4H, CH₂—O), 3.45 (s, 2H, CH₂), 2.07 (m, 6H, CH), 1.92 (d, J=2.8 Hz, 12H, CH₂), 1.67 (t, J=2.8 Hz, 12H, CH₂);

¹³C NMR (100 MHz, CDCl₃): δ=166.8, 154.5, 70.1, 69.0, 65.0, 63.3, 51.1, 42.1, 41.7, 36.6, 29.8;

IR (CHCl₃): 1070, 1132, 1278, 1508, 1720, 2854, 2913 cm⁻¹;

MS (FAB⁺): m/z 284.2 (MH⁺), 306.2 (MNa⁺).

### Preparation of 2,2-fullerenyl-malonic acid bis(adamantan-1-ylcarbamate-diethyleneglycol) ester (Compound 16)

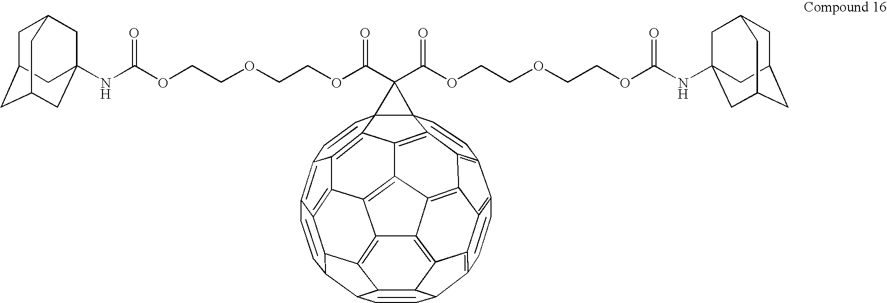

DBU (0.30 grams, 1.96 mmol) was dissolved in toluene (30 ml) and added to a stirred solution of Compound 15 (0.5 gram, 0.79 mmol), C₆₀ (0.57 grams, 0.79 mmol) and I₂ (0.2 grams, 0.79 mmol) in toluene (170 ml), and the mixture was stirred for 36 hours under argon atmosphere at room temperature. Thereafter the reaction mixture was loaded on top of short flash chromatography column packed with silica and eluted with toluene to remove excess fullerene. Further elution with toluene:isopropanol (99:1) gave Compound 16 (0.46 grams, 37% yield) as dark brown solid.

¹H NMR (400 MHz, CDCl₃) δ: 4.83 (broad s, 2H, NH), 4.66 (t, J=4.8 Hz, 4H, CH₂—O—CO), 4.16 (t, J=4.0 Hz, 4H, CH₂—O—CO—NH), 3.88 (t, J=4.8 Hz, 4H, CH₂—O) 3.72 (t, J=4.8 Hz, 4H, CH₂—O), 2.06 (m, 6H, CH), 1.92 (d, J=2.4 Hz, 12H, CH₂), 1.65 (m, 12H, CH₂);

¹³C NMR (100 MHz, CDCl₃): δ=163.5, 154.3, 145.4, 145.30, 145.28, 145.25, 145.0, 144.80, 144.75, 144.70, 144.0, 143.2, 143.14, 143.10, 142.5, 142.3, 141.9, 141.1, 139.2, 72.0, 71.5, 70.7, 70.0, 69.0, 66.3, 63.1, 50.8, 42.1, 41.9, 36.5, 29.6;

IR (CHCl₃): 696, 750, 850, 948, 1103, 1227, 1356, 1457, 1512, 1718, 2359, 2914, 3008 cm⁻¹;

MS (ESI): m/z 1352.56 (M⁺);

λₘₐₓ (CHCl₃): 258, 325, 425, 482, 684 nm.

### Preparation of adamantan-1-yl-carbamic acid poly(n)ethyleneglycol ester wherein average n=10 (Compound 17)

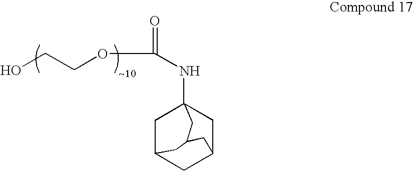

A mixture of 1-adamantylisocyanate (2.0 grams, 11.3 mmol) and a polyethyleneglycol, commonly known as PEG-400, having an average of 10 ethyleneglycol units in each polyethyleneglycol chain and an average molecular weight of about 400 grams per mole (2.4 grams, 22.6 mmol) in dry THF (40 ml) was refluxed for 72 hours under argon atmosphere. After cooling down to room temperature, the solvent was evaporated under reduced pressure and the crude product was purified by flash chromatography on silica using methylene chloride:methanol (9:1) as eluent to give Compound 17 (4.6 grams, 70% yield) as a colorless oil.

Since the starting polyethyleneglycol, namely PEG-400, was comprised of a mixture of polyethyleneglycols of various lengths, an electrospray ionization mass spectrometry (ESI-MS) was found to be particularly useful in analysis of starting material and all subsequent derivatives, including target hybrid compounds.

¹H NMR (400 MHz, CDCl₃): δ=4.68 (broad s, 1H, NH), 4.12 (t, J=4.0 Hz, 2H, CH₂—O—CO), 3.62 (m, 2H, CH₂—O), 2.04 (m, 3H, CH), 1.90 (d, J=2.4 Hz, 6H, CH₂), 1.64 (t, J=2.8 Hz, 6H, CH₂);

¹³C NMR (100 MHz, CDCl₃): δ=154.6, 72.9, 70.89, 70.85, 70.76, 70.62, 70.0, 51.0, 42.1, 36.6, 29.7;

IR (CHCl₃): 1068, 1103, 1279, 1295, 1360, 1456, 1508, 1718, 2911, 3005 cm⁻¹;

MS (ESI⁻): m/z 650 (Ave. MW).

### Preparation of malonic acid bis(adamantan-1-ylcarbamate-poly(n)ethyleneglycol) ester wherein average n=10 (Compound 18)

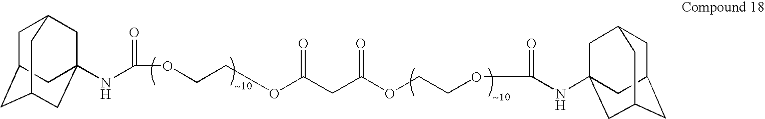

A solution of DCC (1.13 grams, 5.5 mmol) in dry acetonitrile (20 ml) was added dropwise to a solution of malonic acid (0.25 grams, 2.4 mmol) and Compound 17 (3.0 grams, 5.2 mmol) in dry acetonitrile (30 ml) over a time period of 20 minutes under argon atmosphere. The reaction mixture was stirred for additional 24 hours during which a white precipitate was formed. The precipitate was filtered, washed with three portions of methylene chloride (20 ml) and combined organic phase was evaporated under reduced pressure to afford a crude product.

The crude product was purified by flash chromatography on silica using methylene chloride:methanol (95%:5%) as eluent to give Compound 18 (2.29 grams, 78% yield) as a light yellow oil.

¹H NMR (400 MHz, CDCl₃): δ=4.71 (broad s, 2H, NH), 4.21 (t, J=4.8 Hz, 4H, CH₂—O—CO), 4.06 (m, 4H, CH₂—O—CO—NH), 3.63 (t, J=4.8 Hz, 4H, CH₂—O) 3.56 (m, CH₂—O), 3.36 (s, 2H, CH₂) 1.99 (m, 6H, CH), 1.84 (d, J=2.4 Hz, 12H, CH₂), 1.58 (t, J=2.8 Hz, 12H, CH₂);

¹³C NMR (100 MHz, CDCl₃): δ=166.6, 154.4, 70.7, 70.6, 69.8, 68.9, 64.7, 63.2, 50.8, 41.9, 41.4, 36.4, 29.5;

IR (CHCl₃): 1069, 1104, 1245, 1279, 1294, 1456, 1508, 1719, 2911 cm⁻¹;

MS (ESI⁻): m/z 1162 (Ave. MW).

### Preparation of 2,2-fullerenyl-malonic acid bis(adamantan-1-ylcarbamate-poly(n)ethyleneglycol) ester wherein average n=10 (Compound 19)

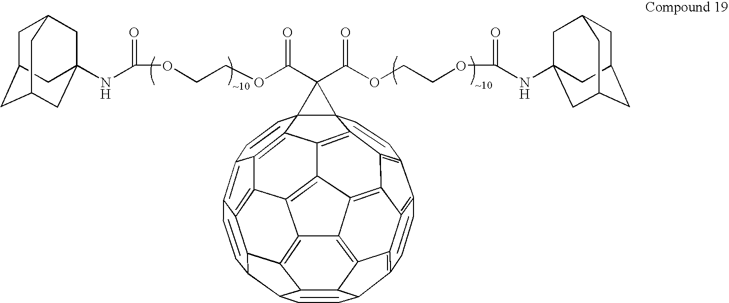

DBU (0.58 grams, 3.8 mmol) was dissolved in toluene (50 ml) and added to a stirred solution of Compound 18 (3.8 gram, 1.55 mmol), C₆₀ (1.1 grams, 1.55 mmol) and I₂ (0.39 grams, 1.55 mmol) in toluene (330 ml), and the mixture was stirred for 72 hours under argon atmosphere at room temperature. Thereafter the reaction mixture was loaded on top of short flash chromatography column packed with silica and eluted with toluene to remove excess fullerene. Further elution with methylene chloride:methanol (9:1) gave Compound 19 (1.36 grams, 45% yield) as viscous dark oil.

As can be seen in FIG. 1, the ESI-MS spectrum obtain for Compound 19 exhibits a mass distribution which is typical for polyethyleneglycol-derived compounds, showing a bell-shaped curve of masses, having a peak (an average) at 1966.5 m/z.

¹H NMR (400 MHz, CDCl₃): δ=4.63 (broad s, 2H, NH), 4.63 (t, J=4.8 Hz, 4H, CH₂—O—CO), 4.11 (t, J=4.0 Hz, 4H, CH₂—O—CO—NH), 3.85 (t, J=4.8 Hz, 4H, CH₂—O) 3.62 (m, ˜60H, CH₂—O), 2.03 (m, 6H, CH), 1.88 (d, J=2.4 Hz, 12H, CH₂), 1.62 (m, 12H, CH₂);

¹³C NMR (100 MHz, CDCl₃): δ=163.6, 154.4, 145.4, 145.33, 145.28, 145.0, 144.8, 144.7, 144.0, 143.24, 143.17, 143.1, 142.3, 142.0, 141.1, 139.2, 72.7, 70.7, 69.9, 68.9, 66.4, 63.2, 61.8, 50.8, 41.9, 36.5, 29.6;

IR (CHCl₃): 521, 699, 751, 855, 949, 1064, 1225, 1290, 1350, 1457, 1509, 1718, 2359, 2801, 2859, 2914, 2964, 3008 cm⁻¹;

MS (ESI): m/z 1966.5 (Average Mw);

λₘₐₓ (CHCl₃): 257, 322, 450, 640, 678 nm.

### Preparation of 2,2-fullerenyl-malonic acid bis(adamantan-1-ylcarbamate-poly(n)ethyleneglycol) ester wherein average n=10 (Compound 20)

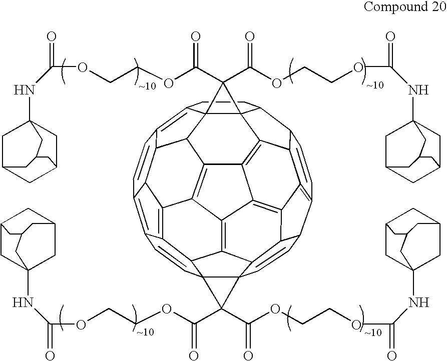

DBU (0.61 grams, 4.0 mmol) was dissolved in toluene (20 ml) and added to a stirred solution of Compound 18 (2.0 gram, 1.55 mmol), C₆₀ (0.54 grams, 0.74 mmol) and I₂ (0.41 grams, 1.6 mmol) in toluene (140 ml), and the mixture was stirred for 72 hours under argon atmosphere at room temperature. Thereafter the reaction mixture was loaded on top of short flash chromatography column packed with silica and eluted with toluene to remove excess fullerene. Further elution with methylene chloride:methanol (96%:4%) gave Compound 20 (1.8 grams, 77% yield) as dark oil.

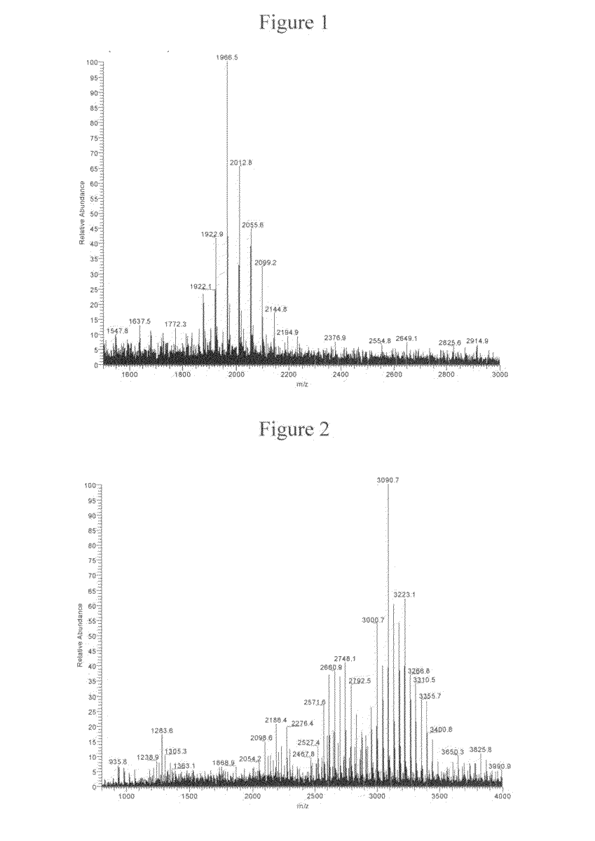

As can be seen in FIG. 2, the ESI-MS spectrum obtain for Compound 20 exhibits a mass distribution which is typical for polyethyleneglycol-derived compounds, showing a bell-shaped curve of masses, having a peak (an average) at 3090.6 m/z.

¹H NMR (400 MHz, CDCl₃): δ=4.66 (broad s, 4H, NH), 4.08 (m, 8H, CH₂—O—CO—NH), 3.59 (m, ˜140H, CH₂—O), 2.00 (m, 12H, CH), 1.86 (m, 24H, CH₂), 1.60 (m, 24H, CH₂);

¹³C NMR (100 MHz, CDCl₃): δ=164.4, 164.3, 155.2, 148.6, 148.3, 148.2, 148.0, 147.7, 147.6, 147.50, 147.78, 147.43, 147.39, 147.2, 147.1, 146.6, 146.5, 146.4, 146.2, 145.7, 145.6, 145.5, 145.42, 145.39, 145.2, 145.1, 145.0, 144.7, 144.6, 144.4, 144.2, 143.9, 143.5, 143.2, 142.9, 142.8, 142.7, 142.6, 142.5, 141.3, 140.3, 139.8, 139.63, 139.61, 73.6, 71.62, 71.59, 71.5, 70.7, 69.8, 69.7, 67.2, 64.1, 62.7, 51.2, 42.8, 37.3, 30.4;

IR (CHCl₃): 685, 749, 789, 853, 946, 1103, 1228, 1288, 1351, 1514, 1716, 2358, 2913, 3005 cm⁻¹;

MS (ESI⁺): m/z 3090.6 (Average Mw);

λₘₐₓ (CHCl₃): 245, 292, 474, 543, 682 nm.

### Aqueous Solubility Assays

One of the basic criteria for bioavailability of the adamantyl-fullerene hybrid compounds of the present invention is their readiness to dissolve in aqueous media. Hence, the hybrid compounds presented herein were assayed for their maximal aqueous solubility using the following method:

20 mg of each of the tested hybrid compounds of the present invention was dissolved in 0.2 ml of DMSO and then diluted with 200 ml, 40 ml, 20 ml and 10 ml of water to obtain a 0.1%, 0.5% 0.1% and 2.0% DMSO content in the aqueous solution, respectively. Each aqueous solution was sonicated for 2 minutes, filtered through 0.2 micron filter and centrifuged at 14,000 rpm for 3 minutes. UV-VIS spectra were obtained thereafter at 476 nm to determine the solubilized fraction of the tested hybrid compound.

The assays were conducted for four exemplary hybrid compounds, namely Compounds 16, 6, 19, wherein n=2, n=4 and n=10 in the poly(n)ethyleneglycol used in their preparation respectively, and Compound 20 in which the fullerene core was doubly substituted with the adamantly-polyethyleneglycol moiety used in Compound 19.

The results of the maximal aqueous solubility assays conducted for the hybrid Compounds 16, 6, 19 and 20 in DMSO solutions are presented in Tables 1 and 2 below.

TABLE 1

| | Maximal aqueous concentration | | | |
|---|---|---|---|---|
| DMSO content in | expressed in units of 10⁻⁵ M | | | |
| final aqueous | Com- | Com- | Com- | Com- |
| solution | pound 16 | pound 6 | pound 19 | pound 20 |
| 0.1% | 0.07 | 1.18 | 1.17 | 1.80 |
| 0.5% | 0.35 | 1.38 | 5.12 | 14.0 |
| 1.0% | 1.40 | 3.14 | 6.97 | 27.0 |
| 2.0% | — | — | — | 51.5 |

TABLE 2

| | |
|---|---|
| DMSO content in | Maximal aqueous concentration |
| final aqueous | expressed in units mg/ml |

| solution | Compound 6 | Compound 20 |
|---|---|---|
| 1.0% | 0.021 | 0.828 |
| 2.0% | 0.036 | 1.592 |

As can be seen in Tables 1 and 2, the maximal aqueous solubility of all adamantyl-fullerene hybrids increased with the increase of the DMSO content in the final tested aqueous solution, increasing more than 2.6 fold with a 10 fold increase in DMSO content in the case of Compound 6, 20 fold in the case of Compound 16, 6 fold in the case of Compound 19, and 15 fold in the case of Compound 20.

As can further be seen in Tables 1 and 2, an increase in the number of ethyleneglycol units in the poly(n)ethyleneglycol moiety from n=2 (diethyleneglycol residue as a bioavailability enhancing moiety), through n=4 (tetraethyleneglycol residue as a bioavailability enhancing moiety) to n=10 (PEG-400 residue as a bioavailability enhancing moiety), was expressed in an increase of solubility, while this factor of increase in solubility diminishes as the content of DMSO increases.

These results, together with the known complications associated with synthetic processes and purification of higher polyethyleneglycols, indicates that a more practical approach for increasing the aqueous solubility of adamantyl-fullerene hybrids would be by multiple substitution of the fullerene residue with adamantly-polyethyleneglycol moieties, rather than the synthesis of mono-substituted fullerenes with longer polyethyleneglycol moieties.

### Biological Activity Assays

As discussed hereinabove, the hybrid compounds of the present invention may be used for treating medical conditions in which neuroprotective activity is beneficial.

Thus, animal models induced with chronic-relapsing autoimmune encephalomyelitis, a medical condition which is ameliorated by neuroprotective activity, were used in order to estimate the degree of neuroprotection offered by the hybrid compounds presented herein.

Experimental autoimmune encephalomyelitis (EAE) has been studied extensively to elucidate mechanisms involved in multiple sclerosis (MS) pathogenesis. Axonal injury begins at disease onset and correlates with the degree of inflammation within lesions, indicating that inflammatory demyelination (loss of the myelin constituting the sheath of a nerve cell) influences axon pathology during relapsing-remitting MS (RR-MS). During secondary progressive MS (SP-MS), chronically demyelinated axons may degenerate due to lack of myelin-derived trophic support. The chronic-relapsing EAE model provides a platform for investigating mechanisms of axon loss and evaluating efficacy of neuroprotective effect of the hybrid compounds presented herein.

More specifically, the hybrid compounds were assayed so as to show that these compounds attenuate the clinical worsening observed in the progressive phase of EAE.

Animal models and Materials

Non-obese diabetic (NOD) mice were purchased from Jackson laboratories. The mice were maintained in viral antibody-free (VAF) facility at Harvard Institutes of Medicine animal care facility and used at 10 weeks of age.

Myelin oligodendrocyte glycoprotein (MOG 35-55) was synthesized at the peptide/protein facility at the center for neurologic disease at BWH, Boston, Mass., USA.

Methods and Results

Mice were immunized S.C. with 150 μg of MOG 35-55 peptide in 4 mg/ml CFA (complete Freund's adjuvant units).

Pertussis toxin was given I.V. (150 ng per mouse) at the time of immunization and 48 hours later. The severity of disease was evaluated daily on the following scale: 0 for no clinical symptoms; 1 for distal tail weakness or tail atonia; 2 for impaired righting reflex and slight hind limb paralysis; 3 for complete paralysis affecting of both hind limbs; 4 for complete paralysis affecting of both hind limbs and fore limb weakness, or moribund state; and 5 for death.

Single injection with MOG 35-55 in NOD mice resulted with first signs of the disease appearing at day 10 (peak at day 16) after immunization, followed by mild clinical impairment in the form of limp tail, impairment righting reflex or hind leg weakness. After recovery from the initial acute attack, there are 2-3 subsequent progressively worsening relapses without full remission in the period therebetween. The relapsing-remitting phase typically advanced to a secondary progressive course characterized by chronic clinical impairment or, in some instances, death.

On day 20 after immunization, mice were randomized into four groups and treated daily with the C3-stereoisomer of trimalonic acid derivative of a C₆₀ fullerene (carboxyfullerene, a highly soluble derivative of C₆₀), two exemplary hybrid compounds according to the present invention, Compound 6 and Compound 20, and the vehicle media (PBS) for control, until termination of the experiment on day 70.

The results of the efficacy assays conducted for the hybrid compounds presented herein in MOG-induced EAE NOD mice are summarized in Table 3 below.

TABLE 3

| | Prevalence | Phase II | | Phase III | Phase IV | | |
|---|---|---|---|---|---|---|---|
| | of Disease | Second Peak | | Third Peak | Secondary Progressive | | Cumulative |
| | with score | Mean Score Disease | | Mean Score Disease | Mean Score Disease | | Score |
| Groups | above 1.5 | day 26 | day 27 | day 31 | day 38 | day 70 | days 21-70 |
| Carboxyfullerene | 8/10 | 1.00 | 0.85 | 1.15 | 1.45 | 2.65 | 87.45 |
| Compound 6 | 9/10 | 0.75* | 0.80 | 0.90* | 1.30 | 2.85 | 90.6 |
| Compound 20 | 4/10 | 0.70* | 0.55* | 0.60* | 0.65* | 1.40* | 53.0* |
| Control | 8/10 | 1.25 | 1.25 | 1.50 | 1.40 | 2.30 | 86.1 |

*p < 0.05 compared to control group

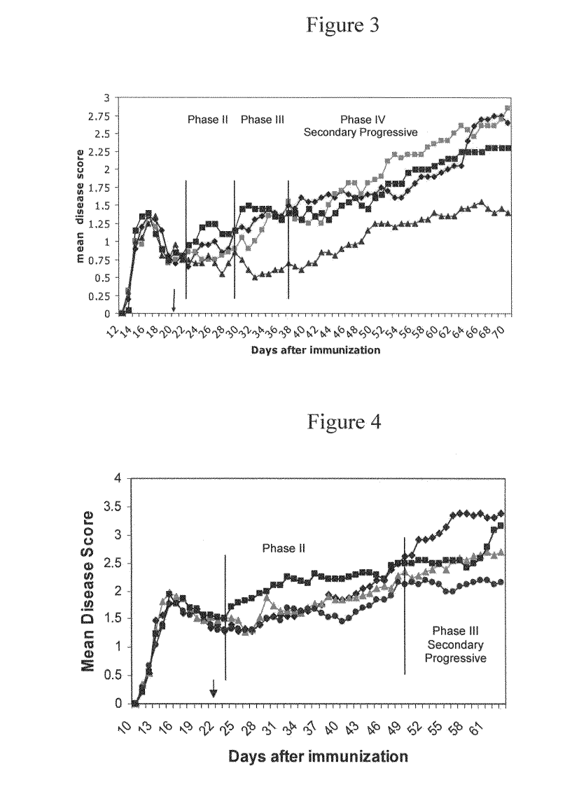

As can be seen in Table 3 and FIG. 3, treatment of EAE in the model mice with Compound 6 and Compound 20, exemplary hybrid compounds of the present invention, clearly reduce the severity of relapsing-remitting EAE in the second phase of the disease in EAE-induced NOD mice, as compared to carboxyfullerene treated and vehicle (PBS control) treated animals.

As can further be seen in Table 3 and FIG. 3, the ameliorating effect of Compound 20 is clearly evident from Phase II of the disease and the secondary progressive to the end of the experiment on day-70, where mice treated with Compound 20 exhibited a significantly low mean disease score of 1.5.

The difference between the derivates of fullerene might be related to their ability to cross the blood brain barrier.

To further assess the effect of hybrid compounds according to the present invention on EAE as compared to the AMPA/kainate antagonist 2,3-dihydroxy-6-nitro-7-sulfamoyl-benzo(f)quinoxaline (NBQX), which was previously reported to ameliorate the disease, NOD mice were randomized into four groups and were treated daily from day 23 after immunization to the end of the experiment with two different doses of Compound 20 (30 or 300 μg/Kg), NBQX (30 mg/Kg) or vehicle (PBS) as control.

The results of the treatment of EAE-induced mice with two doses of Compound 20, an exemplary hybrid compound according to the present invention, compared to treatment of the known drug NBQX are presented in Table 4 below.

TABLE 4

| | | Phase II | | Phase III | |
|---|---|---|---|---|---|
| | | Second Peak | | Secondary Progressive | |
| | | Mean Score Disease | | Mean Score Disease | |
| Groups | Death | day 37 | day 41 | day 62 | day 63 |
| NBQX | 6/14 | 1.75 | 1.88 | 3.31 | 3.38 |
| 30 mg/Kg | | | | | |
| Compound 20 | 0/14 | 1.60* | 1.50* | 2.12* | 2.15* |
| 30 μg/Kg | | | | | |
| Control | 0/14 | 1.77 | 1.88 | 2.65 | 2.69 |
| Compound 20 | 2/14 | 2.25 | 2.25 | 3.08 | 3.17 |
| 300 μg/Kg | | | | | |

*p < 0.05 compared to control group

As be seen in Table 4 and FIG. 4, NBQX showed a tendency to suppress the second phase of EAE but was not as effective as the Compound 20, an exemplary hybrid compound, administered in both 30 and 300 μg/Kg doses, in preventing the chronic disease progression. Furthermore, Compound 20 given daily in dose of 30 μg/Kg significantly protect progression of chronic EAE as compare to higher dose (300 μg/Kg).

The attenuation of EAE progress in model animals was further tested by following pathological findings thereof in 7 μm coronal spinal cord section samples of the tested mice under cryogenic conditions. The axonal pathology analysis was performed on day 63 post-immunization by immunostaining of spinal cord sections. Spinal cord sections from mice were fixed in 4% paraformaldehyde overnight followed by 4.5% sucrose for 4 hours, then 20% sucrose for overnight at 4° C. Spinal cord sections were frozen and stored until used at −80° C.

Histological staining studies of spinal cord sections were preformed according to the Bielschowsky silver staining method, which specifically stains nerve fibers and axons so as to appear in black when observed under an optical microscope, and according to the Luxol fast blue staining method, which specifically stains myelin/myelinated axons so as to stain the myelin in blue-green while the neuron remains pink when observed under an optical microscope. Reduction in the degree of staining expresses damage to the neuron.

The Bielschowsky silver staining was performed as described before in Litchfield and Nagy, Acta Neuropathol (Berl) 2001, 101(1), pp. 17-21. Briefly, spinal cord sections were place in pre-warmed solution of 10% silver nitrate placed in a 40° C. oven and shaken for 15 minutes until sections became light brown in color, and thereafter rinsed in water. The spinal cord sections were thereafter placed back in the same ammonium silver solution and placed in a 40° C. oven for additional 30 minutes and rinse in water followed by dehydration in 95% ethyl alcohol, absolute alcohol and xylene.

The Luxol fast blue staining was perform as described before in Dolcetta et al J Neurosci Res.; 81(4):597-604. Briefly, spinal cord sections were placed in luxol fast blue solution in a 56° C. oven for 16 hours and rinsed with 95% ethyl alcohol and distilled water. Thereafter the spinal cord sections were placed in carbonate solution for 30 seconds and rinsed in water followed by dehydration in 95% ethyl alcohol, absolute alcohol and xylene.

Micrographs of stained spinal cord sections were taken at a magnification of ×20 using a 3-Compatible Camcorder/Digital color video camera (Carl Zeiss).

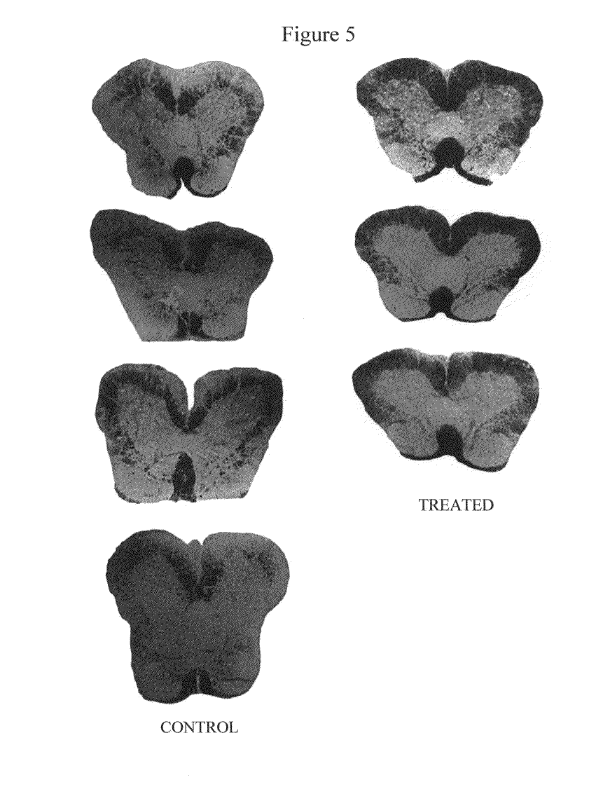

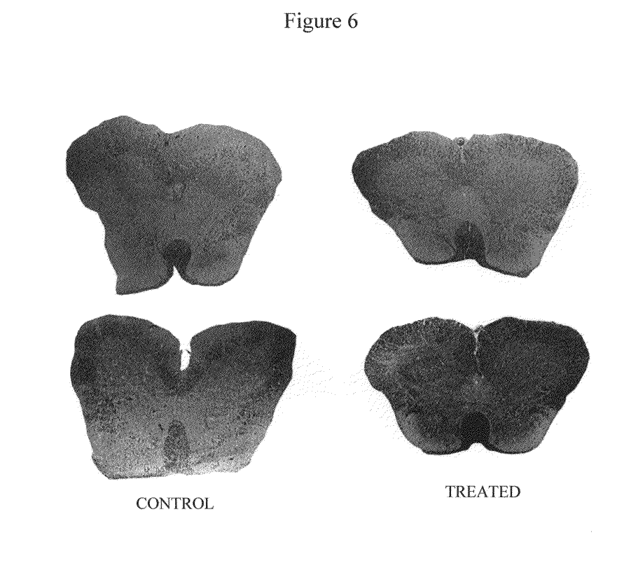

Treatment with Compound 20, initiated after disease onset was shown to attenuate the progression of induced chronic EAE in NOD mice, as expressed in lesser damage caused to the neurons and shown in FIGS. 5 and 6.

FIG. 5 a series of images of sections of the spinal cord of EAE-induced NOD mice after Bielschowsky silver impregnation of axons, showing the effect of treatment of EAE-induced NOD mice with Compound 20 on the extent of EAE-derived axonal damage. As can be seen in FIG. 5, mice treated with Compound 20 exhibited a lower reduction in axonal density in the white matter of the spinal cord (3 images on the right) as compared to untreated control mice (4 images on the left), demonstrating the ameliorating effect of an exemplary hybrid compound presented herein in the treatment of EAE.

FIG. 6 shows a series of images of sections of the spinal cord of EAE-induced NOD mice after staining of axons in the white matter with Luxol fast blue, showing the reduction in demyelination of axons in Compound 20 treated mice (2 images on the right) as compared to untreated control mice (2 images on the left), demonstrating the ameliorating effect of an exemplary hybrid compound presented herein in the treatment of EAE.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A compound comprising a fullerene moiety, at least one glutamate receptor ligand residue and at least one bioavailability enhancing moiety and salts, solvates and hydrates thereof.

2. The compound of claim 1, wherein said at least one bioavailability enhancing moiety comprises a backbone which comprises at least 4 atoms.

3. The compound of claim 1, wherein said backbone comprises at least 5 atoms.

4. The compound of any claim 1, having sufficient aqueous solubility rendering it suitable of being administered in a pharmaceutically effective amount in physiological aqueous media.

5. The compound of claim 4, wherein said pharmaceutically effective amount ranges from about 10 μg per Kg of body weight to about 600 μg per Kg of body weight per day.

6. The compound of claim 1, capable of crossing the blood-brain barrier.

7. The compound of claim 1, having a general Formula I:
FX-Z)ₘ Formula I
wherein:
m is an integer of 1-10;
F is said fullerene moiety;
X is said bioavailability enhancing moiety; and
Z is said at least one glutamate receptor ligand residue.

8. The compound of claim 7, having a general Formula II:
FMX-Z)_{q})ₘ Formula II;
wherein:
M is a first linking moiety; and
q is an integer of 1-10.

9. The compound of claim 7, having a general Formula III:
FMX-Y-Z)_{q})ₘ Formula III
wherein:
Y is a second linking moiety.

10. The compound of claim 7, wherein said bioavailability enhancing moiety has a general formula IV:
-((A)p-D)n Formula IV
wherein:
p is and integer of 1-10;
n is an integer of 1-100;
A is selected from the group consisting of alkyl, alkenyl, cycloalkyl, cycloalkenyl, heteroalicyclic, aryl and heteroaryl;
D is selected from the group consisting of —O—, —S—, —NRa—, —PRa—, —C(═O)O—, —S(═O)O—, —NRaC(═O)—, —OP(═O)O—, —OS(═O)O— or absent; and
Ra is selected from the group consisting of alkyl and hydroxyl.

11. A compound having a general Formula V:
MX-Y-Z)_{q} Formula V
and salts, solvates and hydrates thereof,
wherein:
M is a first linking moiety; and
X is a bioavailability enhancing moiety;
Y is a second linking moiety;
Z is a glutamate receptor ligand residue;
q is an integer of 1-10;
and further wherein: said bioavailability enhancing moiety has a general formula IV:
-((A)p-D)n Formula IV
whereas:
p is and integer of 1-10;
n is an integer of 1-100;
A is selected from the group consisting of alkyl, alkenyl, cycloalkyl, cycloalkenyl, heteroalicyclic, aryl and heteroaryl;
D is selected from the group consisting of —O—, —S—, —NRa—, —PRa—, —C(═O)O—, —S(═O)O—, —NRaC(═O)—, —OP(═O)O—, —O S(═O)O— or absent; and
Ra is selected from the group consisting of alkyl and hydroxyl.

12. The compound of claim 11, wherein said first linking moiety (M) is a malonic acid residue, said bioavailability enhancing moiety (X) is a polyethylene glycol moiety, and q is 2.

13. The compound of claim 11, wherein:
M is a malonic acid residue;
X is poly(ethylene glycol);
Z is an adamantane residue;
Y is C-amide;
A is methylene;
p is 2;
q is 2; and
n is 2, 4 or 10.

14. A method of synthesizing the compound of claim 1, the method comprising:
reacting a bioavailability enhancing moiety and at least one glutamate receptor ligand, to thereby obtain a bioavailability enhancing moiety covalently attached to at least one glutamate receptor ligand residue; and
reacting said bioavailability enhancing moiety covalently attached to said at least one glutamate receptor ligand residue with a fullerene, thereby obtaining the compound.

15. The method of claim 14, wherein said fullerene is covalently attached to at least one bioavailability enhancing moiety via a first linking moiety, the method further comprising, prior to reacting said bioavailability enhancing moiety with said glutamate receptor ligand:
reacting at least one bioavailability enhancing moiety with a first linking moiety, to thereby obtain at least one bioavailability enhancing moiety covalently attached to said first linking moiety.

16. The method of claim 14, wherein said fullerene is covalently attached to at least one bioavailability enhancing moiety via a first linking moiety, the method further comprising, prior to reacting said bioavailability enhancing moiety covalently attached to said at least one glutamate receptor ligand residue with said fullerene:
reacting said bioavailability enhancing moiety covalently attached to said at least one glutamate receptor ligand residue and a first linking moiety, to thereby obtain at least one bioavailability enhancing moiety covalently attached to said at least one glutamate receptor ligand residue at one end and to said first linking moiety at another end.

17. The method of claim 14, wherein said glutamate receptor ligand is attached to said bioavailability enhancing moiety via a second linking moiety.

18. The compound of claim 1, wherein said at least one glutamate receptor ligand residue is selected from the group consisting of an N-methyl-D-aspartic acid (NMDA) receptor ligand residue, an (RS)-2-amino-3-(3-hydroxy-5-methyl-4-isoxazolyl)propionic acid (AMPA) receptor ligand residue and a kainic acid (KA) receptor ligand residue.

19. The compound of claim 18, wherein said glutamate receptor ligand residue is a residue of any of Ligands 1-178 listed in Table A.

20. The compound of claim 19, wherein said at least one glutamate receptor ligand residue is an N-methyl-D-aspartic acid (NMDA) receptor ligand residue.

21. The compound of claim 20, wherein said N-methyl-D-aspartic acid (NMDA) receptor ligand residue is an N-methyl-D-aspartic acid (NMDA) receptor antagonist residue.

22. The compound of claim 21, wherein said N-methyl-D-aspartic acid (NMDA) receptor antagonist residue further comprises a cycloalkyl moiety, said cycloalkyl moiety is selected from the group consisting of an adamantyl, a cubyl, a bicyclo[2.2.1]heptyl, a bicyclo[2.2.2]octyl and a bicyclo[1.1.1]pentyl.

23. The compound of claim 22, wherein said adamatyl is selected from the group consisting of adamantane residue, memantine residue and amantadine residue.

24. The compound of claim 1, wherein said at least one bioavailability enhancing moiety is selected from the group consisting of a poly(alkylene glycol), poly(ethylene imine), poly(vinyl alcohol), poly(methyl vinyl ether), poly(n-isopropyl acrylamide), poly(n,n-dimethyl acrylamide), polyacrylamide and poly(2-hydroxyethyl methacrylate).

25. The compound of claim 24, wherein said poly(alkylene glycol) is selected from the group consisting of poly(ethylene glycol), poly(propylene glycol) and poly(butylene glycol).

26. The compound of claim 25, wherein said poly(alkylene glycol) is poly(ethylene glycol).

27. The compound of claim 8, wherein said first linking moiety is selected from the group consisting of a malonic acid residue, a 5,6,7,8-tetrahydronaphthalene-diol residue, a 5,6,7,8-tetrahydro-naphthalene-diol residue, a pyrrolidine residue, an aziridine residue and a phosphonate residue.

28. The compound of claim 27, wherein said first linking moiety is a malonic acid residue.

29. The compound of claim 9, wherein said second linking moiety is selected from the group consisting of amine, alkyl, alkenyl, cycloalkyl, heteroalicyclic, aryl, heteroaryl, methyleneamine, amine oxide, sulfate, thiosulfate, sulfite, thiosulfite, sulfinate, sulfoxide, sulfonate, S-sulfonamide, N-sulfonamide, disulfide, phosphonate, phosphinyl, phosphine oxide, phosphine sulfide, phosphate, phosphite, thiophosphate, carbonyl, thiocarbonyl, oxime, azo, peroxo, C-carboxylate, O-carboxylate, C-thiocarboxylate, O-thiocarboxylate, N-carbamate, O-carbamate, O-thiocarbamate, N-thiocarbamate, S-dithiocarbamate, N-dithiocarbamate, urea, thiourea, C-amide, N-amide, guanyl, guanidine, hydrazine, hydrazide, thiohydrazide, silyl, siloxy, silaza, silicate, boryl and borate.

30. The compound of claim 29, wherein said second linking moiety is C-amide.

31. The compound of claim 1, wherein said fullerene moiety is selected from the group consisting of a C20 residue, a C24 residue, a C28 residue, a C32 residue, a C34 residue, a C36 residue, a C38 residue, a C40 residue, a C44 residue, a C48 residue, a C50 residue, a C54 residue, a C56 residue, a C60 residue, a C62 residue, a C68 residue, a C70 residue, a C74 residue, a C78 residue, a C80 residue, a C82 residue, a C84 residue, a C86 residue, a C88 residue, a C92 residue, a C94 residue, a C112 residue or a C120 residue.

32. The compound and method of claim 31, wherein said fullerene moiety is a C60 residue.

33. The compound of claim 9, wherein:
Z is an adamantane residue;
X is poly(ethylene glycol);
M is a malonic acid residue;
Y is C-amide;
F is a C60 fullerene moiety;
q is 2; and
m is 1 or 2.

34. The compound of claim 10, wherein:
A is methylene;
p is 2;
n is 2-50.

35. The compound of claim 34, wherein:
m is 1;
n is 2, 4 or 10.

36. The compound of claim 34, wherein:
m is 2;
n is 10.

37. A pharmaceutical composition comprising, as an active ingredient, the compound of claim 1 and a pharmaceutically acceptable carrier.

38. The pharmaceutical composition of claim 37, being packaged in a packaging material and identified in print, in or on said packaging material, for use in the treatment of a medical condition selected from the group consisting of a medical condition in which modulating and/or inhibiting an activity of a glutamate receptor is beneficial, a CNS associated disease, disorder or trauma, an oxidative stress associated disease or disorder, a disease or disorder in which neuroptotection is beneficial, a viral infection, a bacterial infection, cancer and a medical condition at least partially treatable by the compound.

39-40. (canceled)

41. A method of treating a medical condition selected from the group consisting of a medical condition in which modulating and/or inhibiting an activity of a glutamate receptor is beneficial, a CNS associated disease, disorder or trauma, an oxidative stress associated disease or disorder, a disease or disorder in which neuroptotection is beneficial, a viral infection, a bacterial infection, cancer and a medical condition at least partially treatable by the compound of claim 1, the method comprising administering to the subject in need thereof a therapeutically effective amount of the compound.

42. The method of claim 41, wherein said oxidative stress associated disease or disorder is selected from the group consisting of atherosclerosis, an ischemia/reperfusion injury, restenosis, hypertension, cancer, an inflammatory disease or disorder, an acute respiratory distress syndrome (ARDS), asthma, inflammatory bowel disease (IBD), a dermal and/or ocular inflammation, arthritis, metabolic disease or disorder and diabetes.

43. The method of claim 41, wherein said CNS associated disease, disorder or trauma is selected from the group consisting of a neurodegenerative disease or disorder, a stroke, a brain injury and/or trauma, multiple sclerosis, amyotrophic lateral sclerosis, Huntington's disease, Parkinson's disease, Alzheimer's disease, autoimmune encephalomyelitis, AIDS associated dementia, epilepsy, schizophrenia, pain, anxiety, an impairment of memory, a decreased in cognitive and/or intellectual functions, a deterioration of mobility and gait, an altered sleep pattern, a decreased sensory input, a imbalance in the autonomic nerve system, depression, dementia, confusion, catatonia and delirium.
